(19) United States
(12) Patent Application Publication
(10) Pub. No.: US 2018/0031579 A1
(43) Pub. Date: Feb. 1, 2018

(54) METHODS FOR PREDICTING THE RESPONSIVENESS OF A PATIENT AFFECTED WITH MALIGNANT HEMATOLOGICAL DISEASE TO CHEMOTHERAPY TREATMENT AND METHODS OF TREATMENT OF SUCH DISEASE

(71) Applicants: INSERM (INSTITUT NATIONAL DE LA SANTÉ ET DE LA RECHERCHE MÉDICALE), Paris (FR); UNIVERSITÉ PARIS DESCARTES, Paris (FR); CENTRE NATIONAL DE LA RECHERCHE SCIENTIFIQUE (CNRS), Paris (FR); FONDATION IMAGINE, Paris (FR); ASSISTANCE PUBLIQUE-HÔPITAUX DE PARIS (APHP), Paris (FR); UNIVERSITÉ DE NANTES, Nantes (FR); UNIVERSITÉ D'ANGERS, Angers (FR); CENTRE HOSPITALIER UNIVERSITAIRE D'ANGERS, Angers (FR)

(72) Inventors: Ivan CRUZ-MOURA, Paris (FR); Valérie BARDET, Paris (FR); Michaël DUSSIOT, Paris (FR); Thiago TROVATI MACIEL, Paris (FR); Jérôme TAMBURINI, Paris (FR); Norbert IFRAH, Cedex 1 Nantes (FR); Olivier HERMINE, Paris (FR)

(21) Appl. No.: 15/550,394

(22) PCT Filed: Feb. 11, 2016
(86) PCT No.: PCT/EP2016/052935

§ 371 Date: Aug. 11, 2017

(30) Foreign Application Priority Data
Feb. 12, 2015 (EP) 15305207.1

(51) Int. Cl. G01N 33/74; A61K 39/395; C07K 16/22

(52) U.S. Cl. CPC G01N 33/74; C07K 16/22; A61K 39/3955; G01N 2800/52; G01N 2800/22; C07K 2317/76; G01N 2333/51

(57) ABSTRACT

The present invention relates to antagonists of GDF11, for use in the treatment of malignant hematological disease, such as Acute Myeloid Leukemia (AML). The present invention also relates to a method for predicting the responsiveness of a patient affected with malignant haematological disease, such as Acute Myeloid Leukemia, to a chemotherapy treatment.

### FIELD OF THE INVENTION

The present invention relates to antagonists of GDF11, for use in the treatment of malignant hematological disease, such as Acute Myeloid Leukemia (AML). The present invention also relates to a method for predicting the responsiveness of a patient affected with malignant haematological disease, such as Acute Myeloid Leukemia, to a chemotherapy treatment.

### BACKGROUND OF THE INVENTION

Acute myeloid leukemia (AML) is the most frequent form of acute leukemia in adults. This biologically heterogeneous disease results from chromosomal rearrangements and gene mutations¹ that disrupt self-renewal, promote cell survival/proliferation and myeloid differentiation block leading to the accumulation of undifferentiated blasts together with hematopoiesis impairment^{2,3}. When associated with severe infections and bleeding, hematopoiesis impairment can delay the initiation of intensive chemotherapy regimen and thus contribute to patients morbidity and mortality^{4,5}.

AML management remains a challenge and long-term patients' survival is of around 40% despite the use of intensive chemotherapy⁶. This poor prognosis is frequently associated to relapses following chemotherapy-induced remission. Although chemo-resistance is usually observed in relapsing patients it can also be observed following first line chemotherapy. Among newly diagnosed AML primary refractoriness to induction therapy regimen occurs in 10-25% of patients^{7,8} and is defined by the failure of induction/consolidation therapy to achieve an initial complete remission (CR)^{4,9}. The median survival of this group of patients is particularly poor (around 4 months¹⁰). The presence of a past history of a myeloid neoplasm, the development of a therapy-related AML and severe thrombocytopenia have been shown to be associated to a reduction of response to the first cycle of induction therapy¹⁰. Molecular markers of reduced response to induction therapy are poorly investigated. Thus, the identification of biological markers and the understanding of subjacent molecular events leading to chemo-resistance could help to the development of targeted therapies to treat primary refractory and eventually relapsing AML patients.

Therapeutic options proposed for primary refractory AML are limited. Allogeneic stem cell transplantation (SCT) has been shown to be the most effective treatment for these patients¹¹, but toxicity-related to SCT limits its use to medically fit patients which are generally younger than 55 years. Nonetheless, elderly patients are particularly susceptible for therapy refractoriness since events associated with increased chemo-resistance (e.g. poor tolerance to cytotoxic agents, frequency of adverse cytogenetics, the presence of an antecedent hematologic disease, the development of a therapy-related AML, severe thrombocytopenia complication) are frequently found in this group of patients which results in an increased morbidity and mortality^{10,12}. In addition, since the median age for AML diagnosis is of 69 years and since population aging tends to increase in the next years there is an urgent need to the development of new targeted non-toxic therapies adapted for primary refractory elderly AML patients^{13,14}.

Thus, prediction of antitumoral drug response during therapy or even before starting therapy is needed. However, there are no established biomarkers, which may improve the selection of patients who may benefit from the treatment.

Inventors have previously shown that increased expression of Growth differentiation factor 11 (GDF11) induced terminal erythroid differentiation block of β-thalassemia through an autocrine amplification loop involving oxidative stress¹⁵. Moreover, targeting GDF11 by an Actr2A trap (an Actr2A-Fc fusion protein named RAP-011) corrected anemia and limited iron overload in a mouse model of β-thalassemia intermedia¹⁵. Preliminary results from clinical trials are promising showing a good safety profile and an improvement of hemoglobin level and reduction of transfusion burden in thalassemic patients (ClinicalTrials.gov Identifier: NCT01571635). In addition, Suragani et al have shown that GDF11 expression is increased in a mouse model of myelodysplastic syndrome (MDS) and that GDF11 trapping by an Activin receptor 2B (Actr2B) resulted in the correction of anemia in MDS but also in thalassemic mice¹⁵⁻¹⁷. Therefore, GDF11 appeared as a new erythroid regulator involved in the inhibition of terminal erythroid differentiation and thus participating in the pathogenesis of ineffective erythropoiesis of both thalassemia^{15,16} and MDS models¹⁷.

The purpose of the present invention is therefore to address this need by providing a new reliable method for predicting whether a patient affected with a malignant hematological disease is responder or no responder to a chemotherapy treatment and also a new therapeutical target for malignant hematological disease.

### SUMMARY OF THE INVENTION

A first object of the invention relates to a method for predicting the responsiveness of a patient affected with a malignant haematological disease to a chemotherapy treatment, comprising a step of measuring the level of GDF11 in a blood sample from said patient and a step of comparing the level of GDF11 with a control reference value.

A second object of the invention also relates to method for monitoring the effectiveness of treatment of a patient affected with a malignant haematological disease to an anti-cancer treatment comprising a step of measuring the level of GDF11 in a blood sample from said patient and a step of comparing the level of GDF11 with a control reference value.

A third object of the invention also relates to GDF11 antagonist for use in the prevention or treatment of a patient affected with a malignant haematological disease.

The present invention also provides a GDF11 antagonist for use in the treatment of drug resistant cancer or tumor relapse in a patient suffering from malignant haematological disease.

### DETAILED DESCRIPTION OF THE INVENTION

Here the inventors investigated the correlation between GDF11 serum levels and biological findings from AML patients. They found that: 1) GDF11 were increased in AML patients relative to healthy subjects; 2) Increased GDF11 serum levels were not associated with common genetic alterations of AML, with markers of disease burden (such as bone marrow blasts and/or circulating blasts numbers) as well as other pathological features of disease (e.g. anemia and thrombocytopenia); 4) Unexpectedly, they found that increased GDF11 levels segregated with impaired responses to induction chemotherapy and that around 70% of patients presenting GDF11 levels of >40 pg/ml required two or more courses of chemotherapy to achieve CR; 5) Moreover, the use of neutralizing GDF11 antibodies in AML cells resulted in cell apoptosis validating GDF11 targeting as a therapeutic option in AML. Thus, increased GDF11 levels at diagnosis are associated with refractoriness to induction therapy regimen opening new perspectives in AML management.

Methods for Predicting the Responsiveness of a Patient According to the Invention:

A first aspect of the invention consists of a method for predicting the responsiveness of a patient affected with a malignant hematological disease to a chemotherapy treatment, comprising a step of measuring the level of GDF11 in a blood sample from said patient and a step of comparing the expression level of GDF11 with a control reference value.

In a particular embodiment, methods of the invention are suitable for predicting the responsiveness of a patient affected with a malignant hematological disease to a chemotherapy treatment.

As defined herein the term “GDF11” or “Growth differentiation factor 11” (GDF11) also known as “bone morphogenetic protein 11” or “BMP-11” is a protein that in humans is encoded by the GDF11 gene. This BMP group of proteins is characterized by a polybasic proteolytic processing site, which is cleaved to produce a protein containing seven conserved cysteine residues. GDF11 is a myostatin-homologous protein that acts as an inhibitor of nerve tissue growth. GDF11 is a member of the GDF subfamily and shares about 90% amino acid homology with GDF8, also known as myostatin. Both can bind the activin type HA and B receptors and activate the Smad 2/3 signaling pathway. GDF11 plays a big role in development, taking part in the formation of muscle, cartilage, bone, kidney, and nervous system while in adult tissues, GDF11 was detected in the pancreas, intestine, kidney, skeletal muscle, brain and dental pulp. Low amounts of GDF11 can be also found in the circulation. To this date, however, there is no evidence describing a role for GDF11 in malignant hematological disease. One example of wild-type GDF11 human amino acid sequence is provided in SEQ ID NO:1 (NCBI Reference Sequence: NP_005802). One example of nucleotide sequence encoding wild-type GDF11 amino acid sequence of SEQ ID NO:1 is provided in SEQ ID NO:2 (NCBI Reference Sequence: NM_005811).

In one embodiment of the methods defined above, one or more biological markers are quantified together with GDF11.

As used herein, a “biological marker” encompasses any detectable product that is synthesized upon the expression of a specific gene, and thus includes gene-specific mRNA, cDNA and protein.

The various biological markers names specified herein correspond to their internationally recognized acronyms that are usable to get access to their complete amino acid and nucleic acid sequences, including their complementary DNA (cDNA) and genomic DNA sequences. Illustratively, the corresponding amino acid and nucleic acid sequences of each of the biological markers specified herein may be retrieved, on the basis of their acronym names, that are also termed herein “gene symbols”, in the GenBank or EMBL sequence databases. All gene symbols listed in the present specification correspond to the GenBank nomenclature. Their DNA (cDNA and gDNA) sequences, as well as their amino acid sequences are thus fully available to the one skilled in the art from the GenBank database, notably at the following Website address: “http://www.ncbi.nlm.nih.gov/”.

Of course variant sequences of the biological markers may be employed in the context of the present invention, those including but not limited to functional homologues or orthologues of such sequences.

According to the invention, the term “patient”, is intended for a human or non-human mammal affected or likely to be affected with a malignant hematological disease. In preferred embodiment the patient is a human affected or likely to be affected with a malignant hematological disease.

The term “responder” patient, or group of patients, refers to a patient, or group of patients, who show a clinically significant relief in the disease when treated with a chemotherapy treatment, with an increased survival rate or overall survival (OS). Typically for acute leukemia, a responder patient is a patient who obtained complete hematological response. Complete hematological response is defined by the association of the following criteria: Bone marrow blasts<5%; absence of blasts with Auer rods; absence of extramedullary disease; absolute neutrophil count>1.0×10⁹/L (1000/μL); platelet count>100×10⁹/L (100 000/μL) and independence of red cell transfusions. (Döhner H et al, Blood, 2010, p 453). Conversely, a “non responder patient” or group of patients, refers to a patient or group of patients, who do not show a clinically significant relief in the disease when treated with a chemotherapy treatment.

As used herein the term “overall survival” also called “survival rate” means the percentage of people in a study or treatment group who are still alive for a certain period of time after they were diagnosed with or started treatment for a disease, such as cancer. The overall survival rate is often stated as a five-year survival rate, which is the percentage of people in a study or treatment group who are alive five years after their diagnosis or the start of treatment.

By a “chemotherapeuty treatment” is meant a drug that has proved its efficacy for the treatment of cancer, namely a drug having a marketing approval or a drug undergoing clinical or preclinical trial for the treatment of cancer.

Non-limiting examples of chemotherapeutic compounds include, for example, conventional chemotherapeutic, radiotherapeutic and anti-angiogenic agents.

Exemplary chemotherapeutic compounds include alkylating agents, cytotoxic antibiotics such as topoisomerase I inhibitors, topoisomerase II inhibitors, plant derivatives, RNA/DNA antimetabolites, and antimitotic agents. Preferred examples may include, for example, cisplatin (CDDP), carboplatin, cytarabine (Ara-c), azacitidine (5-Azacitidine), decitabine (5-aza-2′-deoxycytidine), procarbazine, mechlorethamine, cyclophosphamide, camptothecin, ifosfamide, melphalan, chlorambucil, chlofarabine, busulfan, nitrosurea, dactinomycin, daunorubicin, doxorubicin, bleomycin, mitoxantrone, plicomycin, mitomycin, etoposide (VP16), tamoxifen, raloxifene, taxol, gemcitabine, navelbine, transplatinum, 5-fluorouracil, vincristin, vinblastin and methotrexate, or any analog or derivative variant of the foregoing.

In preferred embodiment, chemotherapeutic compound are daunorubicin, cytarabine, mitoxantrone, decitabine, and azacitidine.

The term “malignant haematological disease” refer to or describe the pathological condition in mammals that is typically characterized by unregulated haematological cell growth. More precisely, malignant haematological disease according to the invention is due to an unregulated growth of undifferentiated hematopoietic bone marrow cells (hematopoietic stem cell).

As intended herein the expression “hematopoietic stem cell (HSC)” refers to adult multipotent stem cells that give rise to all the blood cell types including for example myeloid lineages (monocytes and macrophages, neutrophils, basophils, eosinophils), erythrocytes, megakaryocytes/platelets, and lymphoid lineages (T-cells, B-cells, NK-cells).

The expression “hematopoietic stem cell malignancy” or “hematopoietic malignancy” according to the invention comprises acute myeloid leukemia (AML), acute lymphoblastic leukemia, Chronic myeloid, lymphoid leukemia, lymphoma and myelodysplastic syndrome (as defined in 2001 WHO classification). Preferably, the hematopoietic malignancy according to the invention is selected from the group consisting of acute myeloid leukaemia. More preferably, the acute myeloid leukemia is an AML of intermediate cytogenic risk group as defined by the revised MRC prognostic classification (Grimwade D, Blood, 2010, p 354). The term “AML of intermediate cytogenic risk group” means AML patient with for instance normal karyotype and/or which expresses the FLT3-ITD mutated tyrosine kinase receptor and/or which expresses NPM1 Nucleophosmin (Nucleolar Phosphoprotein B23, Numatrin) wild type protein and/or which has a genetic abnormalities like translocation t(9:11), . . . (Clinical Features and Risk Stratification of AML see the review Zeisig B B, Cancer Cell, 2012).

The level of the GDF11 may be determined by using standard electrophoretic and immunodiagnostic techniques, including immunoassays such as competition, direct reaction such as immunohistochemistry, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, etc. The reactions generally include revealing labels such as fluorescent, chemiluminescent, radioactive, enzymatic labels or dye molecules, or other methods for detecting the formation of a complex between the antigen and the antibody or antibodies reacted therewith.

For example, determination of the GDF11 level can be performed by a variety of techniques and method any well known method in the art: RIA kits (DiaSorin; IDS, Diasource) Elisa kits (IDS (manual) IDS (adapted on open analyzers) Immunochemiluminescent automated methods (DiaSorin Liaison, Roche Elecsys family, IDS iSYS) (Janssen M J, Steroids, nov 2012).

Control reference values are easily determinable by the one skilled in the art, by using the same techniques as for determining the level of GDF11 in biological samples previously collected from the patient under testing.

A “control reference value” can be a “threshold value” or a “cut-off value”. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. Preferably, the person skilled in the art may compare the GDF11 levels (obtained according to the method of the invention) with a defined threshold value. In one embodiment of the present invention, the threshold value is derived from the GDF11 level (or ratio, or score) determined in a blood sample derived from one or more subjects who are responders to malignant haematological disease treatment. In one embodiment of the present invention, the threshold value may also be derived from GDF11 level (or ratio, or score) determined in a blood sample derived from one or more subjects who are non-responders to malignant haematological disease treatment. Furthermore, retrospective measurement of the GDF11 levels (or ratio, or scores) in properly banked historical subject samples may be used in establishing these threshold values.

Typically a control reference value is 40 pg/ml. Accordingly when the level value found for the GDF11 in the patient tested is inferior to said value it is concluded that the patient tested consists of a responder to the chemotherapy treatment. And when the level value found for the GDF11 in the patient tested is superior to said value it is concluded that the patient tested consists of a non (or bad) responder to the chemotherapy treatment.

Accordingly, when the disease is Acute myeloid leukemia (AML), a preferred responder group of patients that is a group that shows GDF11 levels below the control reference value (e.g. ≦40 pg/ml) before chemotherapy treatment.

After being tested for responsiveness to a chemotherapy treatment, the patients may thus be prescribed with said with a chemotherapy treatment, with reasonable expectations of success. As show in the example, AML patient with a GDF11 levels below the control reference value (e.g. ≦40 pg/ml), before treatment with chemotherapy, need less induction chemotherapy to obtain complete remission (RC) compared to the group of elevated GDF11 levels (>40 pg/ml).

### Monitoring Anti-Cancer Treatments

Monitoring the influence of agents (e.g., drug compounds) on the level of expression of one or more tissue-specific biological markers of the invention can be applied for monitoring the malignant potency of the treated malignant haematological disease of the patient with time. For example, the effectiveness of an agent to affect GDF11 expression can be monitored during treatments of subjects receiving anti-cancer, and especially chemotherapy treatments.

Accordingly a second object of the invention also relates to method for monitoring the effectiveness of treatment of a patient affected with a malignant haematological disease to an anti-cancer treatment comprising a step of measuring the level of GDF11 in a blood sample from said patient and a step of comparing the level of GDF11 with a control reference value.

In a preferred embodiment, the malignant haematological disease is selected from the group consisting of acute myeloid leukemia.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate) comprising the steps of (i) obtaining a pre-administration sample from a subject prior to administration of the agent; (ii) detecting the GDF11 gene expression level; (iii) obtaining one or more post-administration samples from the subject; (iv) detecting GDF11 gene expression level in the post-administration samples; (v) comparing GDF11 gene expression level in the pre-administration sample with the level of expression in the post-administration sample or samples; and (vi) altering the administration of the agent to the subject accordingly. For example, increased expression of GDF11 gene during the course of treatment may indicate ineffective dosage and the desirability of increasing the dosage. Conversely, decreased expression of GDF11 gene may indicate efficacious treatment and no need to change dosage.

In a specific embodiment, the anti-cancer treatment is selected from the group consisting of chemotherapy treatment and/or a GDF11 antagonist.

Because repeated collection of biological samples from the cancer-bearing patient are needed for performing the monitoring method described above, then preferred biological samples consist of blood samples or bone marrow samples susceptible to contain (i) blast cells originating from the patient's malignant haematological disease tissue, or (ii) specific marker expression products synthesized by cells originating from the patients malignant haematological disease tissue, including nucleic acids and proteins.

### Therapeutic Methods and Uses

The present invention provides methods and compositions (such as pharmaceutical compositions) for preventing or treating a malignant haematological disease. The present invention also provides methods and compositions for inhibiting or preventing malignant haematological disease.

In the context of the invention, the term “treatment or prevention” means reversing, alleviating, inhibiting the progress of, or preventing the disorder or condition to which such term applies, or one or more symptoms of such disorder or condition. In particular, the treatment of the disorder may consist in reducing the number of malignant cells. Most preferably, such treatment leads to the complete depletion of the malignant cells.

Preferably, the individual to be treated is a human or non-human mammal (such as a rodent (mouse, rat), a feline, a canine, or a primate) affected or likely to be affected with cancer. Preferably, the individual is a human.

According to a first aspect, the present invention relates to a GDF11 antagonist for use in the prevention or the treatment of a patient affected with a malignant haematological disease.

In one embodiment malignant haematological disease is acute myeloid leukemia.

More preferably, the acute myeloid leukemia is an AML of intermediate cytogenic risk group.

An “GDF11 antagonist” refers to a molecule (natural or synthetic) capable of neutralizing, blocking, inhibiting, abrogating, reducing or interfering with the activities of GDF11 including, for example, reduction or blocking of GDF11 receptor (Actr2A or Actr2B)) activation, reduction or blocking of GDF11 receptor (ActR2A or ActR2B)) downstream molecular signaling. GDF11 antagonists include antibodies and antigen-binding fragments thereof, proteins, peptides, glycoproteins, glycopeptides, glycolipids, polysaccharides, oligosaccharides, nucleic acids, bioorganic molecules, peptidomimetics, pharmacological agents and their metabolites, transcriptional and translation control sequences, and the like. Antagonists also include small molecule inhibitors of a protein and receptor molecules and derivatives which bind specifically to GDF11 thereby sequestering its binding to its GDF11 receptor (ActR2A or ActR2B), such as soluble GDF11 receptors or fusions proteins (including immunoadhesins, e.g ActR2A-Fc molecules), antagonist variants of the protein, siRNA molecules directed to a protein, antisense molecules directed to a protein, aptamers, and ribozymes against a protein. For instance, the GDF11 antagonist may be a molecule which binds to GDF11 or to GDF11 receptor and neutralizes, blocks, inhibits, abrogates, reduces or interferes with the biological activity of GDF11 (such as inducing tumor cell growth). Alternatively, the GDF11 antagonist may be a molecule which binds to GDF11 and neutralizes, blocks, inhibits, abrogates, reduces or interferes with a biological activity of GDF11. More particularly, the GDF11 antagonist according to the invention is an anti-GDF11 antibody, and ActRIIA receptors fusions proteins. More preferably, this GDF11 receptors fusions proteins is ActR2A-Fc such as ACE-011 (sotatercept) or ActR2B-Fc such as ACE-536.

As used herein the term “GDF11 receptor” has its general meaning in the art and refers to Activins Type II receptors which bind GDF11. Two related Activins type II receptors, ActRIIa and ActRIIb, have been identified (Mathews and V ale, 1991, Cell 65:973-982; Attisano et al., 1992, Cell 68: 97-108). Besides GDF II, ActRIIa and ActRIIb can biochemically interact with several other TGF-beta family proteins, including BMP7, Nodal, GDF8, and activin (Yamashita et al., 1995, J. Cell Biol. 130:217-226; Lee and McPherron, 2001, Proc. Natl. Acad. Sci. 98:9306-9311; Yeo and Whitman, 2001, Mol. Cell 7: 949-957; Oh et al., 2002, Genes Dev. 16:2749-54). ALK4 is the primary type I receptor for activins, particularly for activin A, and ALK-7 may serve as a receptor for activins as well, particularly for activin B.

By “biological activity” of a GDF11 is meant inducing tumor cell growth and blocking tumor cell apoptosis

Tests for determining the capacity of a compound to be GDF11 antagonist are well known to the person skilled in the art. In a preferred embodiment, the antagonist specifically binds to GDF11 in a sufficient manner to inhibit the biological activity of GDF11. Binding to GDF11 and inhibition of the biological activity of GDF11 may be determined by any competing assays well known in the art. For example the assay may consist in determining the ability of the agent to be tested as GDF11 antagonist to bind to GDF11. The binding ability is reflected by the Kd measurement. The term “KD”, as used herein, is intended to refer to the dissociation constant, which is obtained from the ratio of Kd to Ka (i.e. Kd/Ka) and is expressed as a molar concentration (M). KD values for binding biomolecules can be determined using methods well established in the art. In specific embodiments, an antagonist that “specifically binds to GDF11” is intended to refer to an inhibitor that binds to human GDF11 polypeptide with a KD of 1 μM or less, 100 nM or less, 10 nM or less, or 3 nM or less. Then a competitive assay may be settled to determine the ability of the agent to inhibit biological activity of GDF11. The functional assays may be envisaged such evaluating the ability to a) induce tumor cell growth and/or b) induce a tumor cell apoptosis (see example with blocking GDF11 antibody and FIG. 6).

The skilled in the art can easily determine whether a GDF11 antagonist neutralizes, blocks, inhibits, abrogates, reduces or interferes with a biological activity of GDF11. To check whether the GDF11 antagonist bind to GDF11 and/or induce tumor cell growth and/or induce a tumor cell apoptosis in the same way than the initially characterized blocking GDF11 antibody and/or binding assay and/or a cell proliferation assay and/or or a cytotoxicity assay and/or apoptosis assay may be performed with each antagonist. For instance apoptosis can be measured with Annexin V-FITC apoptosis detection test and cell proliferation assay can be measured by Uptiblue-proliferation assay) as described in the Examples section.

In one embodiment, the GDF11 antagonist is an inhibitor of the interaction between GDF11 and GDF11 receptor such as Activins type II receptors, ActRIIa and ActRIIb.

The terms “blocking the interaction”, “inhibiting the interaction” or “inhibitor of the interaction” are used herein to mean preventing or reducing the direct or indirect association of one or more molecules, peptides, proteins, enzymes or receptors; or preventing or reducing the normal activity of one or more molecules, peptides, proteins, enzymes, or receptors.

Thus, the term “inhibitor of the interaction between GDF11 and GDF11 receptor” refers to a molecule which can prevent the interaction between GDF11 and GDF11 receptor (ActRIIa and ActRIIb) by competition or by fixing to one of the molecules.

Accordingly, the GDF11 antagonist may be a molecule which binds to GDF11 or GDF11 receptor selected from the group consisting of antibodies, aptamers, polypeptides and small organic molecules.

The skilled in the art can easily determine whether a GDF11 antagonist neutralizes, blocks, inhibits, abrogates, reduces or interferes with a biological activity of GDF11: (i) binding to GDF11 or GDF11 receptor and/or (ii) inducing tumor cell growth and/or (iii) inducing a tumor cell apoptosis.

The present invention also provides a GDF11 antagonist for use in the treatment of drug resistant cancer or tumor relapse in a patient suffering from malignant hematological disease.

“Drug resistance” as used in expressions such as “drug resistant cancer” or “drug resistant cells” or “drug resistant disease” means a circumstance where a disease (e.g., malignant hematological disease) does not respond to a therapeutic agent. Drug resistance can be intrinsic, which means that the disease has never been responsive to the therapeutic agent, or acquired, which means that the disease ceases responding to the agent or agents to which the disease had previously been responsive. For cancers, such therapeutic agent may be a chemotherapeutic drug such as colchicine, vinblastine, doxorubicin, vinca alkaloids, etoposide, taxanes, or other small molecules used in cancer chemotherapy (Cytarabine, Mitoxantrone, Decitabine, Azacitidine and Daunorubicin in AML therapy) Drug resistance may be associated with cancer and other conditions, such as bacterial, viral, protozoal, and fungal diseases.

By “tumor relapse” or “cancer recurrence” is meant the return of cancer after treatment and after a period of time during which the cancer cannot be detected: in a another term it means reappearance of cancer after a disease-free period.

GDF11 Receptor Polypeptide

In one embodiment, the GDF11 antagonist is an isolated GDF11 receptor polypeptide such as Activins type II receptors, ActRIIa and ActRIIb.

As used herein, the term “GDF11 receptor polypeptide” refers to a polypeptide that specifically bind to GDF11 can be used as GDF11 antagonists that bind to and sequester the GDF11 protein (GDF11 Trap), thereby preventing it from signaling.

In a particular embodiment, the GDF11 receptor polypeptide is soluble. A soluble GDF11 receptor polypeptide exerts an inhibitory effect on the biological activity of the GDF11 protein by binding to the protein, thereby preventing it from binding to GDF11 receptor present on the surface of target cells. It is undesirable for a GDF11 receptor polypeptide not to become associated with the cell membrane. In a preferred embodiment, the soluble GDF11 receptor polypeptide lacks any amino acid sequences corresponding to the transmembrane and intracellular domains from the GDF11 receptor from which it is derived.

In a preferred embodiment, said polypeptide is a soluble GDF11 receptor (s GDF11 receptor) or a functional equivalent thereof.

The terms “soluble GDF11 receptor” or “sGDF11 receptor”, as used herein, refer to a polypeptide comprising or consisting of the extracellular region of the GDF11 receptor or a fragment thereof. For example, sGDF11 receptor, particularly Activins type II receptors, ActRIIa and ActRIIb, may include all the extracellular domain of human ActRIIa polypeptides (i.e. a polypeptide comprising or consisting of the amino acid sequence ranging from positions 21-135 of human of human ActRIIa precursor polypeptide (SEQ ID NO: 3 above).

A “functional equivalent of sGDF11 receptor” is a molecule which is capable of binding to GDF11, preferably which is capable of specifically binding to GDF11 such as Activins type II receptors, ActRIIa and ActRIIb. The term “functional equivalent” includes fragments and variants of sGDF11 receptor as above described. As used herein, “binding specifically” means that the biologically active fragment has high affinity for GDF11 but not for control proteins. Specific binding may be measured by a number of techniques such as ELISA, flow cytometry, western blotting, or immunoprecipitation. Preferably, the functionally equivalent specifically binds to GDF11 at nanomolar or picomolar levels. By “biological activity” of a functional equivalent of the extracellular region of the GDF11 receptor such as ActRIIa and ActRIIb is meant i) the capacity to bind to GDF11; and/or (ii) the capacity to induce tumor cell growth arrest; and/or (iii) the capacity to inducing tumor cell apoptosis.

The skilled in the art can easily determine whether a functional equivalent of the extracellular region of the GDF11 receptor is biologically active. To check whether the newly generated polypeptides (i) bind to GDF11 and/or (ii) the capacity to induce tumor cell growth arrest; and/or (iii) the capacity to inducing tumor cell apoptosis, a binding assay, a cell proliferation assay or an apoptosis assay (see in Example for the GDF11 blocking antibody) may be performed with each polypeptide.

Thus, the polypeptide according to the invention encompasses polypeptides comprising or consisting of fragments of the extracellular region of the GDF11 receptor, provided the fragments are biologically active. In the frame of the invention, the biologically active fragment may for example comprise at least 15, 20, 25, 50, 75, 100, 150 or 200 consecutive amino acids of the extracellular region of the GDF11 receptor (such as ActRIIa and ActRIIb).

In preferred embodiments, GDF11 antagonists comprise part of the extracellular domain of an ActRII receptor, such as ActRIIA or ActRIIB, e.g., human ActRIIA or ActRIIB. More specifically, such GDF11 antagonists can be polypeptides comprising the GDF11-binding domain of ActRII, such as ActRIIA or ActRIIB. Without being bound by theory, such GDF11-binding domain comprising polypeptides sequester GDF11 and thereby prevent GDF11 signaling. These GDF11-binding domain comprising polypeptides may comprise all or a portion of the extracellular domain of an ActRII receptor (i.e., all or a portion of the extracellular domain of ActRIIA or all or a portion of the extracellular domain of ActRIIB). In specific embodiments, the extracellular domain of an ActRII receptor is soluble.

In certain embodiments, the GDF11-binding, extracellular domain of an ActRII receptor is mutated relative to the wild-type receptor such that the GDF11-binding, extracellular domain of an ActRII receptor binds with higher affinity to GDF11 than to any other TGFbeta. In particular, the GDF11-binding, extracellular domain of an ActRII receptor is mutated relative to the wild-type receptor such that the GDF11-binding, extracellular domain of an ActRII receptor binds with higher affinity to GDF11 than to Activin A. Such higher affinity can be at least 10%, 25%, 50%, 75%, 100%, 250%, 500%, or 1000% higher than the affinity to the next highest affinity ligand.

In certain embodiments, the GDF11-binding domain comprising polypeptides are linked to an Fc portion of an antibody (i.e., a conjugate comprising an activin-binding domain comprising polypeptide of an ActRII receptor and an Fc portion of an antibody is generated). Without being bound by theory, the antibody portion confers increased stability on the conjugate and/or reduces the patient's immune response against the GDF11 antagonist. In certain embodiments, the GDF11-binding domain is linked to an Fc portion of an antibody via a linker, e.g., a peptide linker.

Examples of such ActRII polypeptide GDF11 antagonists are disclosed in several published patents/applications. For example, ActRIIA polypeptide inhibitors as disclosed in U.S. Pat. No. 7,709,605; U.S. Pat. No. 8,252,900; U.S. Pat. No. 7,960,343; U.S. Pat. No. 7,988,973. Examples of ActRIIB polypeptide inhibitors that bind several TGF-beta ligands are known in the art and disclosed, for example, in U.S. Pat. No. 8,138,143, U.S. Pat. No. 8,058,229 and U.S. Pat. No. 7,947,646. Examples of ActRIIB polypeptide inhibitors that specifically bind GDF8 and GDF11 are disclosed in U.S. Pat. No. 7,842,663. Examples of ActRIIB antagonists that specifically bind GDF11 are disclosed in U.S. Pat. No. 8,216,997. Clinical ActRII traps include AMG-745, ACE-031, ACE-536 and ACE-011.

In one embodiment, the polypeptides of the invention may comprise a tag. A tag is an epitope-containing sequence which can be useful for the purification of the polypeptides. It is attached to by a variety of techniques such as affinity chromatography, for the localization of said peptide or polypeptide within a cell or a tissue sample using immunolabeling techniques, the detection of said polypeptide by immunoblotting etc. Examples of tags commonly employed in the art are the GST (glutathion-S-transferase)-tag, the FLAG™-tag, the Strep-Tag™, V5 tag, myc tag, His tag (which typically consists of six histidine residues), etc.

In another embodiment, the polypeptides of the invention may comprise chemical modifications improving their stability and/or their biodisponibility. Such chemical modifications aim at obtaining polypeptides with increased protection of the polypeptides against enzymatic degradation in vivo, and/or increased capacity to cross membrane barriers, thus increasing its half-life and maintaining or improving its biological activity. Any chemical modification known in the art can be employed according to the present invention. Such chemical modifications include but are not limited to:
replacement(s) of an amino acid with a modified and/or unusual amino acid, e.g. a replacement of an amino acid with an unusual amino acid like Nle, Nva or Orn; and/or
modifications to the N-terminal and/or C-terminal ends of the peptides such as e.g. N-terminal acylation (preferably acetylation) or desamination, or modification of the C-terminal carboxyl group into an amide or an alcohol group;
modifications at the amide bond between two amino acids: acylation (preferably acetylation) or alkylation (preferably methylation) at the nitrogen atom or the alpha carbon of the amide bond linking two amino acids;
modifications at the alpha carbon of the amide bond linking two amino acids such as e.g. acylation (preferably acetylation) or alkylation (preferably methylation) at the alpha carbon of the amide bond linking two amino acids.
chirality changes such as e.g. replacement of one or more naturally occurring amino acids (L enantiomer) with the corresponding D-enantiomers;
retro-inversions in which one or more naturally-occurring amino acids (L-enantiomer) are replaced with the corresponding D-enantiomers, together with an inversion of the amino acid chain (from the C-terminal end to the N-terminal end);
azapeptides, in which one or more alpha carbons are replaced with nitrogen atoms; and/or
betapeptides, in which the amino group of one or more amino acid is bonded to the 0 carbon rather than the a carbon.

Another strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain.

Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications.

Those of skill in the art are aware of PEGylation techniques for the effective modification of drugs. For example, drug delivery polymers that consist of alternating polymers of PEG and tri-functional monomers such as lysine have been used by VectraMed (Plainsboro, N.J.). The PEG chains (typically 2000 daltons or less) are linked to the a- and e-amino groups of lysine through stable urethane linkages. Such copolymers retain the desirable properties of PEG, while providing reactive pendent groups (the carboxylic acid groups of lysine) at strictly controlled and predetermined intervals along the polymer chain. The reactive pendent groups can be used for derivatization, cross-linking, or conjugation with other molecules. These polymers are useful in producing stable, long-circulating pro-drugs by varying the molecular weight of the polymer, the molecular weight of the PEG segments, and the cleavable linkage between the drug and the polymer. The molecular weight of the PEG segments affects the spacing of the drug/linking group complex and the amount of drug per molecular weight of conjugate (smaller PEG segments provides greater drug loading). In general, increasing the overall molecular weight of the block co-polymer conjugate will increase the circulatory half-life of the conjugate. Nevertheless, the conjugate must either be readily degradable or have a molecular weight below the threshold-limiting glomular filtration (e.g., less than 60 kDa).

In addition, to the polymer backbone being important in maintaining circulatory half-life, and biodistribution, linkers may be used to maintain the therapeutic agent in a pro-drug form until released from the backbone polymer by a specific trigger, typically enzyme activity in the targeted tissue. For example, this type of tissue activated drug delivery is particularly useful where delivery to a specific site of biodistribution is required and the therapeutic agent is released at or near the site of pathology. Linking group libraries for use in activated drug delivery are known to those of skill in the art and may be based on enzyme kinetics, prevalence of active enzyme, and cleavage specificity of the selected disease-specific enzymes. Such linkers may be used in modifying the protein or fragment of the protein described herein for therapeutic delivery.

Alternatively, a nucleic acid encoding a polypeptide of the invention (such as sGDF11 receptor) or a vector comprising such nucleic acid or a host cell comprising such expression vector may be used in the prevention or treatment of a malignant haematological disease.

Nucleic acids of the invention may be produced by any technique known per se in the art, such as, without limitation, any chemical, biological, genetic or enzymatic technique, either alone or in combination(s).

Expression vectors of the invention are well known in the art (since they are easily constructed using conventional methods or are commercially available) and are disclosed below (see the section “Inhibitors of GDF11 gene expression”).

In another particular embodiment, the polypeptide is a GDF11 receptor fusion protein. As used herein, “GDF11 receptor fusion protein” means a protein comprising a soluble GDF11 receptor polypeptide fused to a heterologous polypeptide (i.e. polypeptide derived from an unrelated protein, for example, from an immunoglobulin protein).

As used herein, the terms “fused” and “fusion” are used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the correct translational reading frame of the original ORFs. Thus, a recombinant fusion protein is a single protein containing two or more segments that correspond to polypeptides encoded by the original ORFs (which segments are not normally so joined in nature.) Although the reading frame is thus made continuous throughout the fused segments, the segments may be physically or spatially separated by, for example, in-frame linker sequence.

As used herein, the term “fusion protein” means a protein comprising a first polypeptide linearly connected, via peptide bonds, to a second, polypeptide.

As used herein, the term “GDF11 receptor fusion protein” refers to a polypeptide comprising the extracellular region of the GDF11 receptor or a fragment thereof fused to heterologous polypeptide. The GDF11 receptor fusion protein will generally share at least one biological property in common with s GDF11 receptor (as described above).

An example of a GDF11 receptor fusion protein is a GDF11 receptor immunoadhesin. As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM.

The term “GDF11 receptor immunoadhesin” is used interchangeably with the term “GDF11 receptor-1-immunoglobulin chimera”, and refers to a chimeric molecule that combines at least a fragment of an GDF11 receptor molecule (native or variant) with an immunoglobulin sequence. For instance, the GDF11 receptor immunoadhesin comprises the extracellular domain (ECD) of GDF11 receptor or a fragment thereof sufficient to bind to GDF11.

In a preferred embodiment, the GDF11 receptor immunoadhesin comprises a polypeptide comprising or consisting of the amino acid sequence ranging from positions 21-135 of SEQ ID NO: 3 (ActRIIA polypeptide precursor) and an immunoglobulin sequence.

In another preferred embodiment, the GDF11 receptor immunoadhesin comprises a polypeptide comprising or consisting of the amino acid sequence ranging from positions 19-134 of SEQ ID NO: 4 (ActRIIB polypeptide precursor) and an immunoglobulin sequence.

The immunoglobulin sequence preferably, but not necessarily, is an immunoglobulin constant domain (Fc region). Immunoadhesins can possess many of the valuable chemical and biological properties of human antibodies. Since immunoadhesins can be constructed from a human protein sequence with a desired specificity linked to an appropriate human immunoglobulin hinge and constant domain (Fc) sequence, the binding specificity of interest can be achieved using entirely human components. Such immunoadhesins are minimally immunogenic to the patient, and are safe for chronic or repeated use. In one embodiment, the Fc region is a native sequence Fc region. In another embodiment, the Fc region is a variant Fc region. In still another embodiment, the Fc region is a functional Fc region. The GDF11 receptor portion and the immunoglobulin sequence portion of the GDF11 receptor immunoadhesin may be linked by a minimal linker. The immunoglobulin sequence preferably, but not necessarily, is an immunoglobulin constant domain. The immunoglobulin moiety in the chimeras of the present invention may be obtained from IgG1, IgG2, IgG3 or IgG4 subtypes, IgA, IgE, IgD or IgM, but preferably IgG1 or IgG3.

As used herein, the term “Fc region” is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof.

A “functional Fc region” possesses an “effector function” of a native sequence Fc region. Exemplary “effector functions” include Clq binding; complement dependent cytotoxicity; Fc receptor binding; antibody-dependent cell-mediated cytotoxicity (ADCC); phagocytosis; down regulation of cell surface receptors (e.g. B cell receptor; BCR), etc.

A “native sequence Fc region” comprises an amino acid sequence identical to the amino acid sequence of an Fc region found in nature. Native sequence human Fc regions include a native sequence human IgGi Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

The polypeptides of the invention may be produced by any suitable means, as will be apparent to those of skill in the art. In order to produce sufficient amounts of a sGDF11 receptor or functional equivalents thereof, or a GDF11 receptor fusion protein such as a GDF11 receptor immunoadhesin for use in accordance with the present invention, expression may conveniently be achieved by culturing under appropriate conditions recombinant host cells containing the polypeptide of the invention. Preferably, the polypeptide is produced by recombinant means, by expression from an encoding nucleic acid molecule. Systems for cloning and expression of a polypeptide in a variety of different host cells are well known.

When expressed in recombinant form, the polypeptide is preferably generated by expression from an encoding nucleic acid in a host cell. Any host cell may be used, depending upon the individual requirements of a particular system. Suitable host cells include bacteria mammalian cells, plant cells, yeast and baculovirus systems. Mammalian cell lines available in the art for expression of a heterologous polypeptide include Chinese hamster ovary cells. HeLa cells, baby hamster kidney cells and many others. Bacteria are also preferred hosts for the production of recombinant protein, due to the ease with which bacteria may be manipulated and grown. A common, preferred bacterial host is E. coli.

Antibody

In another embodiment, the GDF11 antagonist is an antibody (the term including antibody fragment or portion) that can block the interaction of GDF11 receptor with GDF11.

In preferred embodiment, the GDF11 antagonist may consist in an antibody directed against the GDF11 receptor or GDF11, in such a way that said antibody impairs the binding of a GDF11 to GDF11 receptor (“neutralizing antibody”).

Then, for this invention, neutralizing antibody of GDF11 or the GDF11 receptor are selected as above described (for their capacity to (i) bind to GDF11 or GDF11 receptor and/or (ii) induce tumor cell growth and/or (iii) induce a tumor cell apoptosis).

In one embodiment of the antibodies or portions thereof described herein, the antibody is a monoclonal antibody. In one embodiment of the antibodies or portions thereof described herein, the antibody is a polyclonal antibody. In one embodiment of the antibodies or portions thereof described herein, the antibody is a humanized antibody. In one embodiment of the antibodies or portions thereof described herein, the antibody is a chimeric antibody. In one embodiment of the antibodies or portions thereof described herein, the portion of the antibody comprises a light chain of the antibody. In one embodiment of the antibodies or portions thereof described herein, the portion of the antibody comprises a heavy chain of the antibody. In one embodiment of the antibodies or portions thereof described herein, the portion of the antibody comprises a Fab portion of the antibody. In one embodiment of the antibodies or portions thereof described herein, the portion of the antibody comprises a F(ab′)2 portion of the antibody. In one embodiment of the antibodies or portions thereof described herein, the portion of the antibody comprises a Fc portion of the antibody. In one embodiment of the antibodies or portions thereof described herein, the portion of the antibody comprises a Fv portion of the antibody. In one embodiment of the antibodies or portions thereof described herein, the portion of the antibody comprises a variable domain of the antibody. In one embodiment of the antibodies or portions thereof described herein, the portion of the antibody comprises one or more CDR domains of the antibody.

As used herein, “antibody” includes both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. The antibody may be a human or nonhuman antibody. A nonhuman antibody may be humanized by recombinant methods to reduce its immunogenicity in man.

Antibodies are prepared according to conventional methodology. Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with antigenic forms of GDF11. The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes.

Briefly, the recombinant GDF11 may be provided by expression with recombinant cell lines. Recombinant form of GDF11 may be provided using any previously described method.

Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods, as described (Coding, Monoclonal Antibodies: Principles and Practice: Production and Application of Monoclonal Antibodies in Cell Biology, Biochemistry and Immunology, 3rd edition, Academic Press, New York, 1996). Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The Fc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

This invention provides in certain embodiments compositions and methods that include humanized forms of antibodies. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference. The above U.S. Pat. Nos. 5,585,089 and 5,693,761, and WO 90/07861 also propose four possible criteria which may used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3A of the CDRs in a three dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization.

In one embodiment of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody retains a similar antigenic specificity as the original antibody. However, using certain methods of humanization, the affinity and/or specificity of binding of the antibody may be increased using methods of “directed evolution”, as described by Wu et al., J. Mol. Biol. 294:151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans.

In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

Thus, as will be apparent to one of ordinary skill in the art, the present invention also provides for F(ab′) 2 Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The various antibody molecules and fragments may derive from any of the commonly known immunoglobulin classes, including but not limited to IgA, secretory IgA, IgE, IgG and IgM. IgG subclasses are also well known to those in the art and include but are not limited to human IgG1, IgG2, IgG3 and IgG4.

In another embodiment, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb.

Examples of anti-GDF8/11 antibodies are disclosed, for example, in several published patents and applications, for example, U.S. Pat. Nos. 8,066,995; 7,320,789 (murine monoclonal antibody JA-16, ATCC Deposit No. PTA-4236); U.S. Pat. No. 7,655,763 (e.g., human monoclonal antibodies Myo29 (Stamulumab) (ATCC Deposit No. PTA-4741), Myo22 (ATCC Deposit No. PTA-4740), Myo28 (ATCC Deposit No. PTA-4739)); U.S. Pat. No. 7,261,893 and US Patent Application No. 20110293630 (Ser. No. 13/115,170). Examples of monoclonal antibodies that can be used with the methods provided herein include antibodies from LifeSpan Biosciences Inc., Seattle, Wash., with catalog numbers LS-C121127, LS-C138772, LS-C105098 (available); antibodies available from Santa Cruz Biotechnology, Inc., Santa Cruz, Calif., with catalog number (X-19): sc-81952; antibodies available from Abcam, with catalog number:ab71347, ab56644 and antibodies available from Sigma-Aldrich Co. LLC, with product number: WH0010220M3. The skilled artisan can use routine technologies to use the antigen-binding sequences of these antibodies (e.g., the CDRs) and generate humanized antibodies for treatment of AML as disclosed herein.

Aptamer

In another embodiment, the GDF11 antagonist is an aptamer directed against GDF11 receptor or GDF11. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996).

Then, for this invention, neutralizing aptamers of GDF11 are selected as above described (for their capacity to (i) bind to GDF11 or GDF11 receptor and/or (ii) induce tumor cell growth and/or (iii) induce a tumor cell apoptosis).

Small Organic Molecule

In another embodiment, the GDF11 antagonist is a small organic molecule. As used herein, the term “small organic molecule” refers to a molecule of size comparable to those organic molecules generally sued in pharmaceuticals. The term excludes biological macromolecules (e.g.; proteins, nucleic acids, etc.); preferred small organic molecules range in size up to 2000 Da, and most preferably up to about 1000 Da.

Inhibitor of GDF11 Gene Expression

In still another embodiment, the GDF11 antagonist is an inhibitor of GDF11 gene expression. An “inhibitor of expression” refers to a natural or synthetic compound that has a biological effect to inhibit the expression of a gene. Therefore, an “inhibitor of GDF11 gene expression” denotes a natural or synthetic compound that has a biological effect to inhibit the expression of GDF11 gene.

In a preferred embodiment of the invention, said inhibitor of GDF11 gene expression is a siRNA, an antisense oligonucleotide, a nuclease or a ribozyme.

Inhibitors of GDF11 gene expression for use in the present invention may be based on antisense oligonucleotide constructs. Anti-sense oligonucleotides, including anti-sense RNA molecules and anti-sense DNA molecules, would act to directly block the translation of GDF11 mRNA by binding thereto and thus preventing protein translation or increasing mRNA degradation, thus decreasing the level of GDF11, and thus activity, in a cell. For example, antisense oligonucleotides of at least about 15 bases and complementary to unique regions of the mRNA transcript sequence encoding GDF11 can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of GDF11 gene expression for use in the present invention. GDF11 gene expression can be reduced by using small double stranded RNA (dsRNA), or a vector or construct causing the production of a small double stranded RNA, such that GDF11 gene expression is specifically inhibited (i.e. RNA interference or RNAi). Methods for selecting an appropriate dsRNA or dsRNA-encoding vector are well known in the art for genes whose sequence is known (e.g. see Tuschi, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Examples of said siRNAs against human GDF11 include, but are not limited to, those purchased by Life technologies, Origene, Santa Cruz, Qiagen.

Inhibitors of GDF11 gene expression for use in the present invention may be based nuclease therapy (like Talen or Crispr).

The term “nuclease” or “endonuclease” means synthetic nucleases consisting of a DNA binding site, a linker, and a cleavage module derived from a restriction endonuclease which are used for gene targeting efforts. The synthetic nucleases according to the invention exhibit increased preference and specificity to bipartite or tripartite DNA target sites comprising DNA binding (i.e. TALE or CRISPR recognition site(s)) and restriction endonuclease target site while cleaving at off-target sites comprising only the restriction endonuclease target site is prevented.

Restriction endonucleases (also called restriction enzymes) as referred to herein in accordance with the present invention are capable of recognizing and cleaving a DNA molecule at a specific DNA cleavage site between predefined nucleotides. In contrast, some endonucleases such as for example Fokl comprise a cleavage domain that cleaves the DNA unspecifically at a certain position regardless of the nucleotides present at this position. Therefore, preferably the specific DNA cleavage site and the DNA recognition site of the restriction endonuclease are identical. Moreover, also preferably the cleavage domain of the chimeric nuclease is derived from a restriction endonuclease with reduced DNA binding and/or reduced catalytic activity when compared to the wildtype restriction endonuclease.

According to the knowledge that restriction endonucleases, particularly type II restriction endonucleases, bind as a homodimer to DNA regularly, the chimeric nucleases as referred to herein may be related to homodimerization of two restriction endonucleases subunits. Preferably, in accordance with the present invention the cleavage modules referred to herein have a reduced capability of forming homodimers in the absence of the DNA recognition site, thereby preventing unspecific DNA binding. Therefore, a functional homodimer is only formed upon recruitment of chimeric nucleases monomers to the specific DNA recognition sites. Preferably, the restriction endonuclease from which the cleavage module of the chimeric nuclease is derived is a type llP restriction endonuclease. The preferably palindromic DNA recognition sites of these restriction endonucleases consist of at least four or up to eight contiguous nucleotides. Preferably, the type llP restriction endonucleases cleave the DNA within the recognition site which occurs rather frequently in the genome, or immediately adjacent thereto, and have no or a reduced star activity. The type llP restriction endonucleases as referred to herein are preferably selected from the group consisting of: Pvull, EcoRV, BamHl, Bcnl, BfaSORF1835P, BfiI, Bgll, Bglll, BpuJl, Bse6341, BsoBl, BspD6I, BstYl, Cfr101, Ecl18kl, EcoO109l, EcoRl, EcoRll, EcoRV, EcoR124l, EcoR124ll, HinP11, Hincll, Hindlll, Hpy99l, Hpy188l, Mspl, Munl, Mval, Nael, NgoMIV, Notl, OkrAl, Pabl, Pacl, PspGl, Sau3A1, Sdal, Sfil, SgrAl, Thal, VvuYORF266P, Ddel, Eco57l, Haelll, Hhall, Hindll, and Ndel.

Other nuclease for use in the present invention are disclosed in WO 2010/079430, WO2011072246, WO2013045480, Mussolino C, et al (Curr Opin Biotechnol. 2012 October; 23(5):644-50) and Papaioannou I. et al (Expert Opinion on Biological Therapy, March 2012, Vol. 12, No. 3: 329-342) all of which are herein incorporated by reference.

Ribozymes can also function as inhibitors of GDF11 gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of GDF11 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Antisense oligonucleotides, siRNAs and ribozymes useful as inhibitors of GDF11 gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides, siRNAs and ribozymes of the invention may be delivered in vivo alone or in association with a vector. In its broadest sense, a “vector” is any vehicle capable of facilitating the transfer of the antisense oligonucleotide, siRNA or ribozyme nucleic acid to the cells and preferably cells expressing GDF11. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in KRIEGLER (A Laboratory Manual,” W.H. Freeman C.O., New York, 1990) and in MURRY (“Methods in Molecular Biology,” vol. 7, Humana Press, Inc., Cliffton, N.J., 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g., SANBROOK et al., “Molecular Cloning: A Laboratory Manual,” Second Edition, Cold Spring Harbor Laboratory Press, 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

### Method of Preventing or Treating Cancer

The present invention further contemplates a method of preventing or treating malignant haematological disease in a subject comprising administering to the subject a therapeutically effective amount of a GDF11 antagonist.

The present invention further also provides a method of preventing or treating drug resistant cancer or tumor relapse in a subject suffering from malignant haematological disease comprising administering to the subject a therapeutically effective amount of an GDF11 antagonist.

In one embodiment malignant haematological disease is acute myeloid leukemia.

More preferably, the acute myeloid leukemia is AML of intermediate cytogenic risk group.

In one aspect, the present invention provides a method of inhibiting tumor growth in a subject comprising administering a therapeutically effective amount of an GDF11 antagonist.

By a “therapeutically effective amount” of a GDF11 antagonist as above described is meant a sufficient amount of the antagonist to prevent or treat a malignant haematological disease. It will be understood, however, that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidential with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

### Pharmaceutical Compositions of the Invention

The GDF11 antagonist as described above may be combined with pharmaceutically acceptable excipients, and optionally sustained-release matrices, such as biodegradable polymers, to form therapeutic compositions.

Accordingly, the present invention relates to a pharmaceutical composition comprising a GDF11 antagonist according to the invention and a pharmaceutically acceptable carrier.

The present invention also relates to a pharmaceutical composition for use in the prevention or treatment of malignant haematological disease comprising a GDF11 antagonist according to the invention and a pharmaceutically acceptable carrier.

In one embodiment malignant haematological disease is acute myeloid leukemia.

More preferably, the acute myeloid leukemia is AML of intermediate cytogenic risk group.

The present invention further relates to a pharmaceutical composition for preventing or treatment of drug resistant cancer or tumor relapse in a subject suffering from malignant haematological disease comprising a GDF11 antagonist according to the invention and a pharmaceutically acceptable carrier.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In therapeutic applications, compositions are administered to a patient already suffering from a disease, as described, in an amount sufficient to cure or at least partially stop the symptoms of the disease and its complications. An appropriate dosage of the pharmaceutical composition is readily determined according to any one of several well-established protocols. For example, animal studies (for example on mice or rats) are commonly used to determine the maximal tolerable dose of the bioactive agent per kilogram of weight. In general, at least one of the animal species tested is mammalian. The results from the animal studies can be extrapolated to determine doses for use in other species, such as humans for example. What constitutes an effective dose also depends on the nature and severity of the disease or condition, and on the general state of the patient's health.

In therapeutic treatments, the antagonist contained in the pharmaceutical composition can be administered in several dosages or as a single dose until a desired response has been achieved. The treatment is typically monitored and repeated dosages can be administered as necessary. Compounds of the invention may be administered according to dosage regimens established whenever inactivation of GDF11 is required.

The daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 10 mg/kg of body weight per day. It will be understood, however, that the specific dose level and frequency of dosage for any particular patient may be varied and will depend upon a variety of factors including the activity of the specific compound employed, the metabolic stability, and length of action of that compound, the age, the body weight, general health, sex, diet, mode and time of administration, rate of excretion, drug combination, the severity of the particular condition, and the host undergoing therapy.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

The appropriate unit forms of administration include forms for oral administration, such as tablets, gelatine capsules, powders, granules and solutions or suspensions to be taken orally, forms for sublingual and buccal administration, aerosols, implants, forms for subcutaneous, intramuscular, intravenous, intranasal or intraocular administration and forms for rectal administration.

In the pharmaceutical compositions of the present invention, the active principle is generally formulated as dosage units containing from 0.5 to 1000 mg, preferably from 1 to 500 mg, more preferably from 2 to 200 mg of said active principle per dosage unit for daily administrations.

When preparing a solid composition in the form of tablets, a wetting agent such as sodium laurylsulfate can be added to the active principle optionally micronized, which is then mixed with a pharmaceutical vehicle such as silica, gelatine, starch, lactose, magnesium stearate, talc, gum arabic or the like. The tablets can be coated with sucrose, with various polymers or other appropriate substances or else they can be treated so as to have a prolonged or delayed activity and so as to release a predetermined amount of active principle continuously.

A preparation in the form of gelatin capsules is obtained by mixing the active principle with a diluent such as a glycol or a glycerol ester and pouring the mixture obtained into soft or hard gelatine capsules.

A preparation in the form of a syrup or elixir can contain the active principle together with a sweetener, which is preferably calorie-free, methyl-paraben and propylparaben as an antiseptic, a flavoring and an appropriate color.

The water-dispersible powders or granules can contain the active principle mixed with dispersants or wetting agents, or suspending agents such as polyvinyl-pyrrolidone, and also with sweeteners or taste correctors.

Rectal administration is effected using suppositories prepared with binders which melt at the rectal temperature, for example cacao butter or polyethylene glycols.

Parenteral, intranasal or intraocular administration is effected using aqueous suspensions, isotonic saline solutions or sterile and injectable solutions which contain pharmacologically compatible dispersants and/or wetting agents, for example propylene glycol, butylene glycol, or polyethylene glycol.

Thus a cosolvent, for example an alcohol such as ethanol or a glycol such as polyethylene glycol or propylene glycol, and a hydrophilic surfactant such as Tween® 80, can be used to prepare an aqueous solution injectable by intravenous route. The active principle can be solubilized by a triglyceride or a glycerol ester to prepare an oily solution injectable by intramuscular route.

Transdermal administration is effected using multilaminated patches or reservoirs into which the active principle is in the form of an alcoholic solution.

Administration by inhalation is effected using an aerosol containing for example sorbitan trioleate or oleic acid together with trichlorofluoromethane, dichlorotetrafluoroethane or any other biologically compatible propellant gas.

The active principle can also be formulated as microcapsules or microspheres, optionally with one or more carriers or additives.

Among the prolonged-release forms which are useful in the case of chronic treatments, implants can be used. These can be prepared in the form of an oily suspension or in the form of a suspension of microspheres in an isotonic medium.

The active principle can also be presented in the form of a complex with a cyclodextrin, for example .alpha.-, .beta.- or .gamma.-cyclodextrin, 2-hydroxypropyl-.beta.-cyclodextrin or methyl-.beta.-cyclodextrin.

### Combination Therapies of the Invention

In other embodiments, the GDF11 antagonist may be administered to a subject with an appropriate additional therapeutic agent useful in prevention or treatment of the condition from which the patient suffers or is susceptible to; examples of such agents include a chemotherapeutic agent, an immunomodulatory agent, a hormonal agent, an immunotherapeutic agent (like antibody anti CD33, . . . ), etc.

The administration of the GDF11 antagonist and the other therapeutic agent, (e.g., a chemotherapeutic agent) can be carried out simultaneously, e.g., as a single composition or as two or more distinct compositions using the same or different administration routes. Alternatively, or additionally, the administration can be done sequentially, in any order. Alternatively, or additionally, the steps can be performed as a combination of both sequentially and simultaneously, in any order. In certain embodiments, intervals ranging from minutes to days, to weeks to months, can be present between the administrations of the two or more compositions. For example, the additional therapeutic agent may be administered first, followed by the GDF11 antagonist. However, simultaneous administration or administration of the GDF11 antagonist first is also contemplated.

Accordingly, in one aspect, the present invention relates to a pharmaceutical composition comprising a GDF11 antagonist according to the invention and an additional therapeutic agent.

In another aspect, the present invention relates to a kit-of-part composition comprising a GDF11 antagonist according to the invention and an additional therapeutic agent.

In still another aspect, the present invention malignant haematological disease comprising a GDF11 antagonist according to the invention and an additional therapeutic agent.

Also provided, is a pharmaceutical composition for use in the treatment or prevention of drug resistant cancer or tumor relapse in a subject suffering from malignant haematological disease comprising a GDF11 antagonist according to the invention and an additional therapeutic agent.

Combination GDF11 Antagonist with a Chemotherapeutic Compound:

It has been shown that when two or more different treatments are combined, the treatments may work synergistically and allow reduction of dosage of each of the treatments, thereby reducing the detrimental side effects exerted by each compound at higher dosages. In other instances, malignancies that are refractory to a treatment may respond to a combination therapy of two or more different treatments.

Non-limiting examples of chemotherapeutic compounds which can be used in combination treatments of the present invention include, for example, conventional chemotherapeutic, radiotherapeutic and anti-angiogenic agents.

Exemplary chemotherapeutic compounds include alkylating agents, cytotoxic antibiotics such as topoisomerase I inhibitors, topoisomerase II inhibitors, plant derivatives, RNA/DNA antimetabolites, and antimitotic agents. Preferred examples may include, for example, cisplatin (CDDP), carboplatin, cytarabine (Ara-c), azacitidine (5-Azacitidine), decitabine (5-aza-2′-deoxycytidine), procarbazine, mechlorethamine, cyclophosphamide, camptothecin, ifosfamide, melphalan, chlorambucil, chlofarabine, busulfan, nitrosurea, dactinomycin, daunorubicin, doxorubicin, bleomycin, mitoxantrone, plicomycin, mitomycin, etoposide (VP16), tamoxifen, raloxifene, taxol, gemcitabine, navelbine, transplatinum, 5-fluorouracil, vincristin, vinblastin and methotrexate, or any analog or derivative variant of the foregoing.

In preferred embodiment, chemotherapeutic compound are daunorubicin, cytarabine, mitoxantrone, decitabine, and azacitidine.

The invention will be further illustrated by the following figures and examples. However, these examples and figures should not be interpreted in any way as limiting the scope of the present invention.

### FIGURES

FIG. 1: gdf11 mRNA Levels are Higher in AML Compared with Normal HSC or Controls.

(A) Retrospective transcriptomic analysis of datasets GSE 13159 and GSE17054 show that gdf11 expression is higher in AML subgroups presenting recurrent cytogenetic alterations (AML with AML1-ETO n=39, AML with inv 16 n=28, APL n=37, AML with 11q23 n=38) than in normal hematopoietic stem cells (HSC, n=8), p<10-4. (B) gdf11 expression is higher in AML (n=404) than in non AML control patients (n=138)(GSE 15061), p<10-4.

FIG. 2: GDF11 is overexpressed in a sub-population of patients with AML.

(A) Serum GDF11 concentrations in AML patients (n=195) versus controls (n=55), means+/−SEM. (B) Serum GDF11 concentrations>7 in AML patients (n=29) and controls (n=14), means+/−SEM.

FIG. 3: GDF11 levels are not correlated with full blood count parameters in AML.

Hemoglobin levels (A), platelets (B) and absolute blast cells counts (C) were correlated with GDF11 in 140 AML patients enrolled in the LAMIR2006 clinical trial.

FIG. 4: GDF11 overexpression is not associated with a recurrent genotype.

Prevalence of the most frequent recurrent mutations in 132 AML patients with low (<40 pg/mL) or elevated (≧40 pg/mL) GDF11 levels.

FIG. 5: GDF11 overexpression is associated with induction therapy failure and primary chemoresistance.

(A) Patients with elevated GDF11 level (≧40 pg/mL, n=18) harbor at early response assessment (D15) after induction therapy more frequently blastic bone marrow compared to patients with lower GDF11 (n=148; P=0.016). (B) Patients with elevated GDF11 level (n=16) need more chemotherapy courses to obtain complete remission (P=0.001).

FIG. 6: GDF11 blockade induces apoptosis and growth arrest in AML cell lines.

(A) Dose-response apoptosis in 4 AML cell lines (MOLM 14, THP1, HL 60 and MV 4-11). (B) Uptiblue-proliferation assay in MV4-11 and THP1 cell lines treated with anti GDF11 antibody (ab71347). (C) Dose-response apoptosis in MV4-11 AML cell line with 4 anti-GDF11 antibodies.

FIG. 7: GDF11 blockade induces apoptosis and growth arrest in primary AML cell.

Primary AML cells from eight different patients (SU 1 to 8) were cultured with growing concentrations of anti GDF11 antibody (ab71347).

### EXAMPLE

Material & Methods

Patients and Controls

Sera and blast cells from patients with AML were obtained from the GOELAMS' biobank after acceptance of the study by the scientific committee. 174 patients enrolled in the LAMIR2006 clinical trial (patients aged between 18-60 years and presenting de novo AML with intermediate risk, NCT00860639) and 21 patients enrolled in the CBF trial (patients aged between 18-60 years and presenting de novo or therapy-related Core Binding Factor (CBF) AML, NCT00428558) were included in this study. 55 sera were obtained from healthy controls after informed consent.

Statistics

All statistics were performed with GraphPad Prism 5 (GraphPad Software Inc.) All tests used were two-tailed tests and a P-value<0.05 was considered as significant.

Cell Culture and Cytotoxicity Assays

MOLM-14, MV4-11, HL60, THP1 AML cells lines and primary AML cells were cultured in alpha MEM medium supplemented with 5% FCS and glutamine. Cell lines were cultured at 250 000/ml in 24-well plates during 48h with various concentrations of anti-human GDF11 antibodies: ab71347, ab56645 (Abcam), WH0010220M3 (Sigma) and sc-6884 (Santa Cruz Biotechnology). Primary AML cells were cultured at 3 10⁶/ml in 48-well plates during 5 days. Apoptosis was measured with Annexin V-FITC apoptosis detection kit (Becton Dickinson Pharmingen) coupled with TO-PRO-3 (Life Technologies) staining according to manufacturers' instructions. For Uptiblue cytotoxicity bioassay (Interchim), cells were cultured at 50 000/ml in 96-well plates during 48h and fluorescence was measured with a Victor X4 multilabel plate reader (Perkin Elmer).

Elisa

GDF11 levels were measured using Elisa kit (Mybiosource) according to manufacturer's instructions.

Results

GDF11 is Overexpressed in a Sub-Population of Patients with AML.

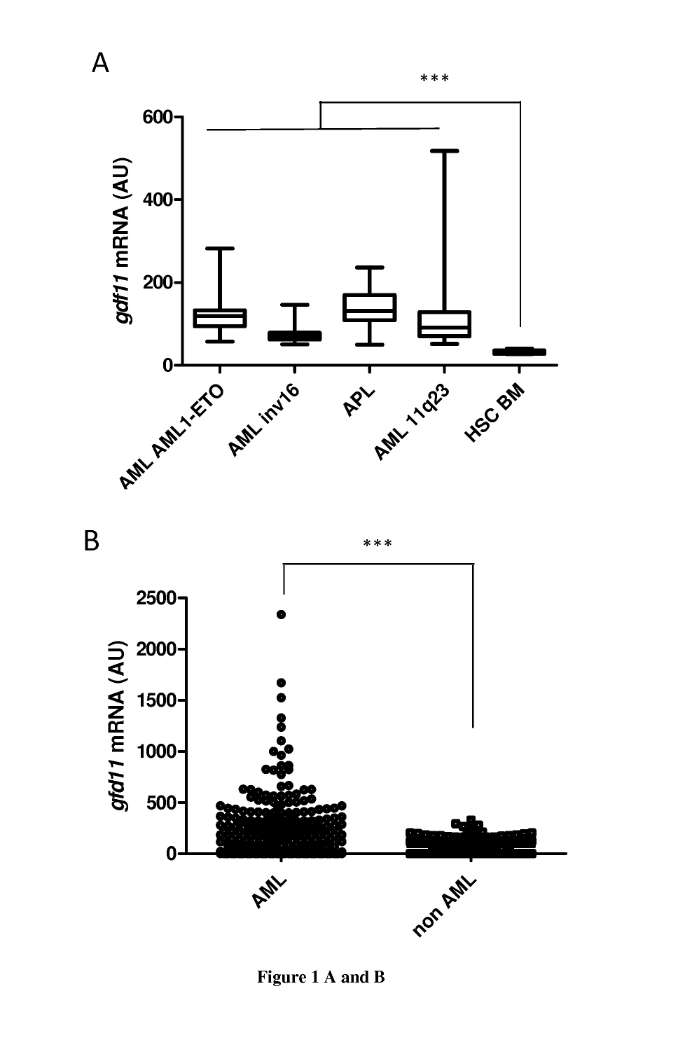

We investigated the levels of GDF11 expression in AML patients. Through an investigation of distinct AML subgroups presenting recurrent cytogenetic alterations found in the disease (http://servers.binf.ku.dk/hemaexplorer) we found that gdf11 expression was higher in AML with AML1-ETO, APL, AML with inv(16)/t(16;16) and AML with t(11q23) subgroups compared to healthy hematopoietic stem cells (FIG. 1A; p<10-4). Further retrospective analysis of transcriptomic data from a large set of AML patients (GSE 15061) revealed that gdf11 expression was increased in AML patients compared to non-AML controls (FIG. 1B, p<10-4). Therefore, gdf11 levels are increased in samples from AML compared to healthy normal HSCs or non-AML controls.

To further confirm results from gene expression analysis we searched to detect circulating GDF11 levels in favorable and intermediate cytogenetic risk-AML patients at diagnosis (n=195 sera from the GOELAMS' biobank-Groupe Ouest-Est des Leucemies Aigues et Maladies du Sang—protocols NCT00860639 and NCT00428558). Healthy individuals with no declared hematological disorder were used as controls (n=55 sera). GDF11 levels were determined by a commercial ELISA kit (Mybiosource).

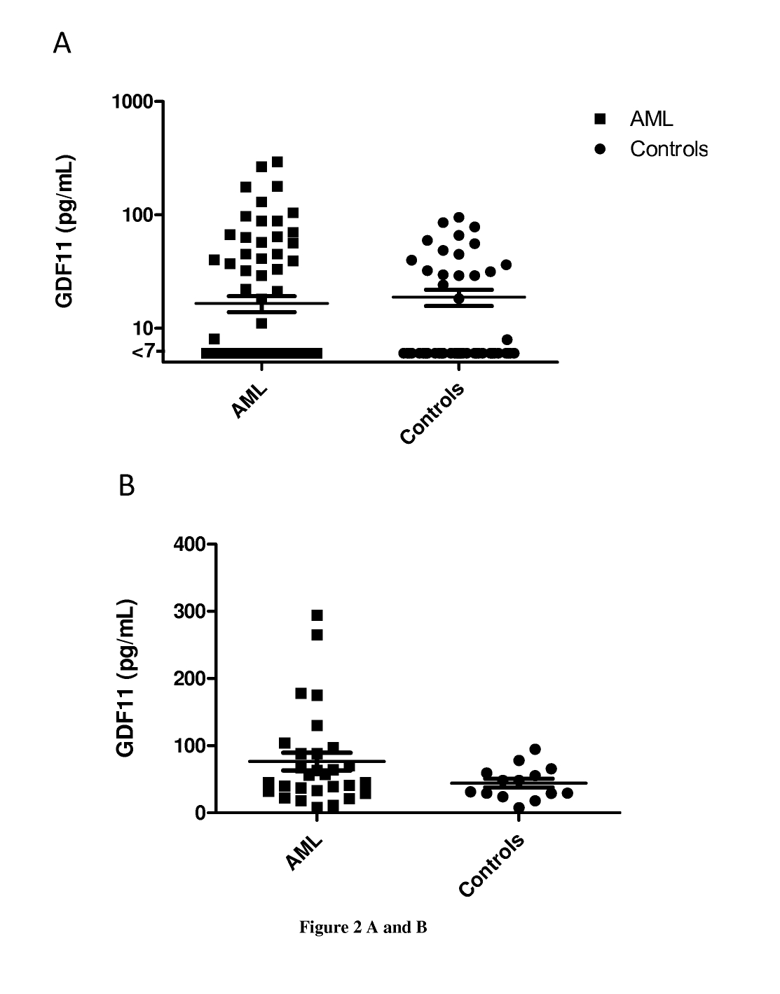

Detectable GDF11 levels (>7pg/ml) were found in 16% AML patients and did not differed in frequency from healthy subjects (FIG. 2A). Absence of differences between the frequencies of individuals with detectable GDF11 levels suggests that GDF11 expression is not a feature of AML. When comparing subject groups presenting detectable GDF11 (>7pg/ml) observed that the mean level of circulating GDF11 was significantly elevated in AML patients (7613; range, 8-294 pg/mL) compared with healthy controls (44±6; range, 8-95 pg/mL) (p=0.035; FIG. 2B). Therefore, circulating GDF11 levels are increased in AML patients compared to controls.

GDF11 Overexpression is not Associated to Anemia of AML or Disease Burden

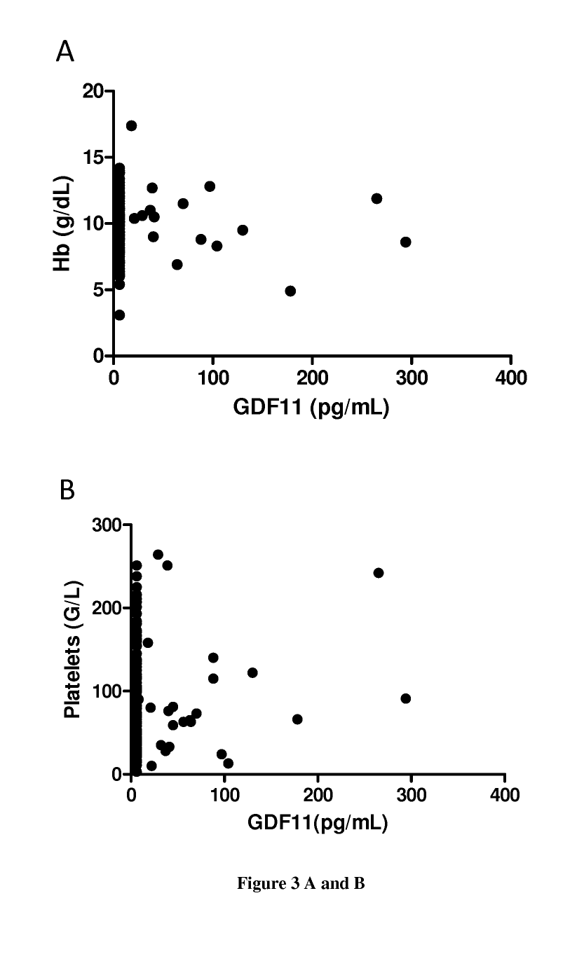

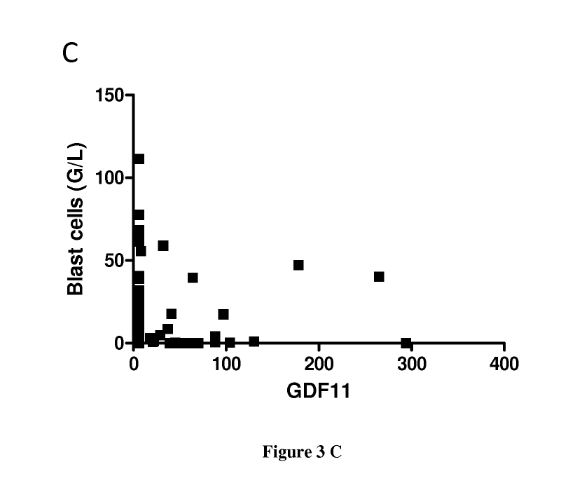

AML patients frequently develop anemia which can be associated with alterations of erythroid lineage differentiation. Since increased GDF11 levels have been shown to promote ineffective erythropoiesis in both MDS and thalassemia¹⁸ We asked whether GDF11 overexpression could be associated with anemia of AML. Analysis of sera obtained from intermediate cytogenetic risk-AML patients at diagnosis (N=140) with different degrees of anemia or normal hemoglobin levels (hemoglobin levels ranging from 6 to 14 g/L) shown that there was no correlation between anemia and circulating GDF11 levels (FIG. 3A). Further analysis showed the absence of correlation between circulating GDF11 levels and thrombocytopenia (FIG. 3B) or the number of circulating blasts (FIG. 3C) suggesting that GDF11 is not correlated with disease progression leukemic burden.

GDF11 Overexpression does not Correlates with Common AML Somatic Mutations Found in NK-AML

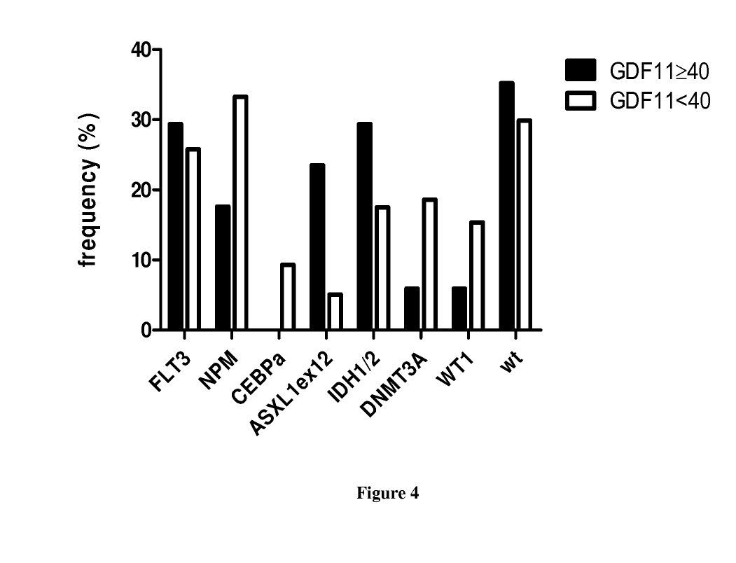

Since increased GDF11 serum levels were present in a subpopulation of intermediate cytogenetic risk AML patients which are mainly normal karyotypes (80%) we searched for correlations between increased circulating levels of GDF11 and recurrent somatic mutations present in this patient group. Elevated circulating GDF11 levels were not associated with FLT3, NPM1, CEBPa, ASXL1, IDH1, IDH2, WT1 or DNMT3A mutations (FIG. 4). Therefore, GDF11 overexpression is independent of mutational status of AML patients.

GDF11 Overexpression Correlates with Impaired Remission Induction.

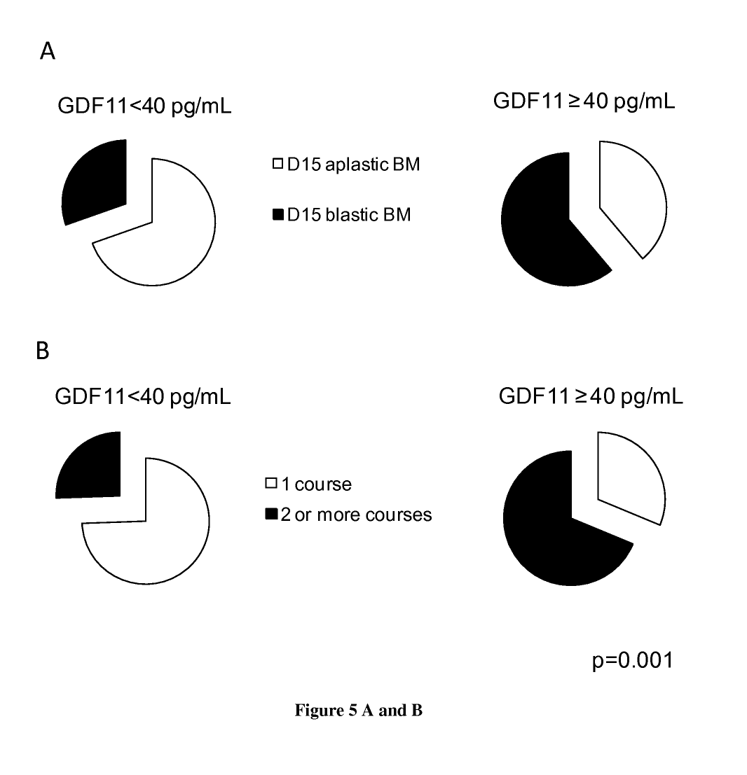

We next searched for the correlation of increased GDF11 levels and response to induction chemotherapy. Whereas patients presenting circulating GDF11 levels of <40 pg/ml (GDF11 low, cutoff determined following receiver operating characteristic (ROC) curve analysis of combined ELISA results) presented around 70% of aplastic bone marrows at early response assessment (Day 15) following induction chemotherapy, the subgroup of patients presenting GDF11≧40 pg/mL (GDF11 high) presented around 61% of blastic bone marrows following induction therapy (FIG. 5A). Therefore, GDF11 high AML bear features associated with reduced responses to induction chemotherapy regimen.

We next analyzed the number of chemotherapy courses necessary to achieve complete remission (CR) in GDF11 low and GDF11 high subgroups. We observed that around 69% of patients of the GDF11 low subgroup achieved CR following 1 course of intensive chemotherapy regimen whereas 72% of patients in the GDF11 high group received two or more courses of chemotherapy to achieve CR. Therefore, increased circulating GDF11 levels segregated with impaired chemotherapy responses in AML patients. Thus, increased GDF11 levels at diagnosis are associated with impaired remission induction.

Neutralization of GDF11 Induces Cell Apoptosis in AML Cell Lines.

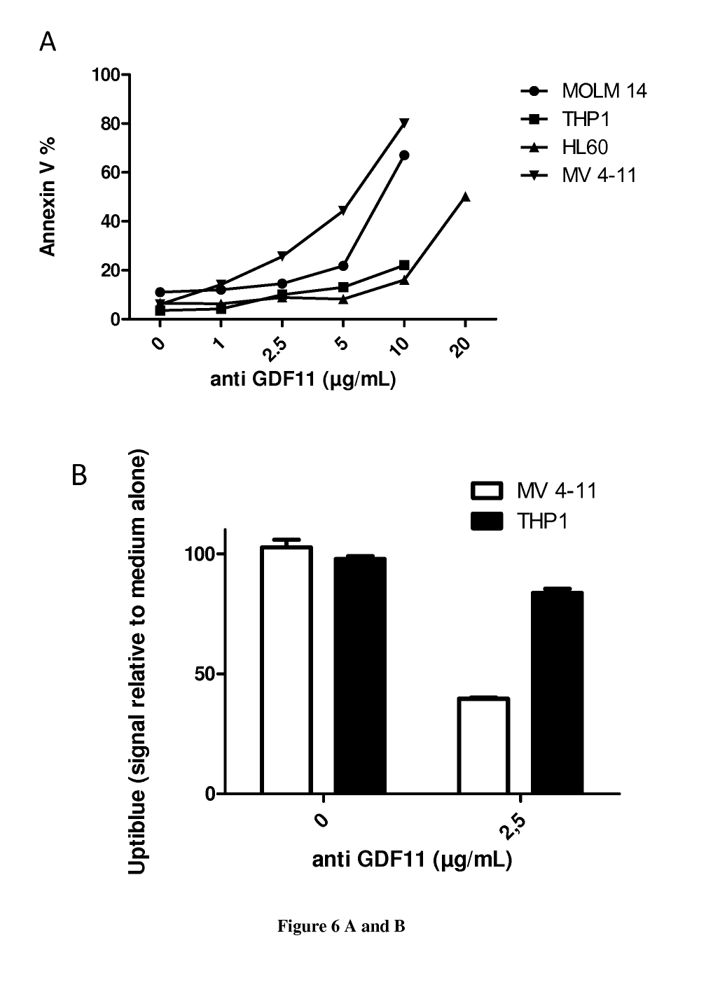

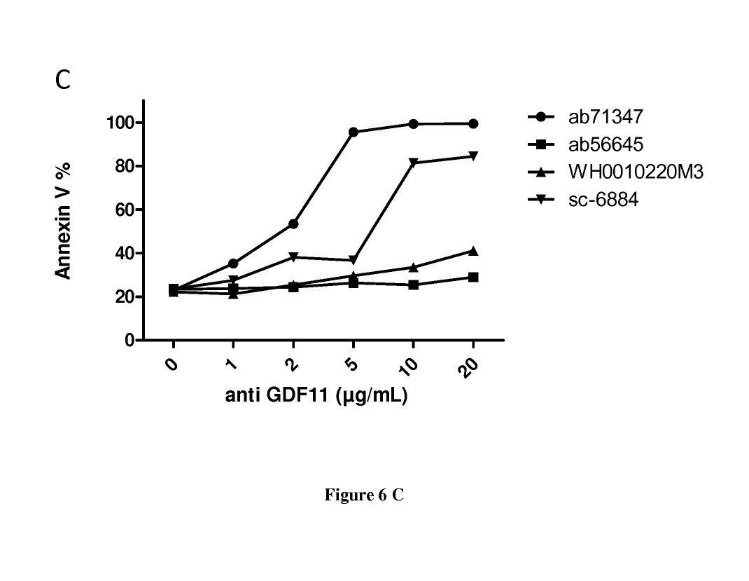

We searched for the cellular consequences of GDF11 neutralization in AML cells. AML cells (MOLM-14, THP1, HL60 and MV4-11 cell lines) were cultured with growing doses of GDF11 blocking antibodies and apoptosis was evaluated by Annexin V staining. All AML cells were sensitive to anti-GDF11 blocking antibodies suggesting that GDF11 expression in required for AML cell growth (FIG. 6A). In addition, anti-GDF11 antibodies also induce a cell growth arrest (FIG. 6B). Further use of different commercial antibodies confirmed previous data showing data blocking antibodies could induce cell apoptosis (FIG. 6C).

Neutralization of GDF11 Induces Cell Apoptosis in Primary AML Blasts.

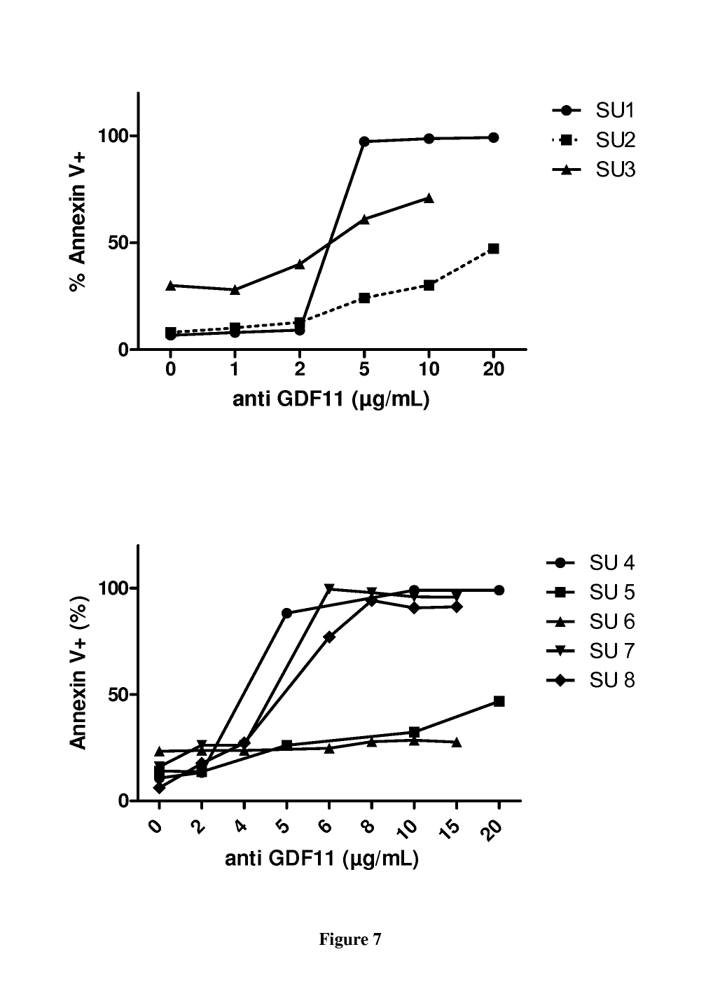

Primary AML blasts from eight different patients were cultured with growing doses of GDF11 blocking antibodies and apoptosis was evaluated (FIG. 7). Among the eight samples, only one sample was insensitive to anti-GDF11 blocking antibodies (subject 6), samples corresponding to subjects 5 and 6 showed intermediate sensitivity to GDF11 blockade (around 50% apoptosis) whereas samples corresponding to subjects 1,3,4,7 and 8 were highly sensitive to GDF11 inhibition.

### CONCLUSION

Primary refractoriness to induction chemotherapy therapy regimen occurs in 10-25% of newly diagnosed acute myeloid leukemia patients (AML) and is associated with dismal prognosis⁴. Here we show that increased GDF11 serum levels in intermediate cytogenetic risk AML is associated with impaired responses to chemotherapy regimen. GDF11 levels do not segregate with the most frequent somatic mutations found in the disease therefore defining a new patient subgroup with possible consequence to disease management. Indeed GDF11 appeared as a new marker predicting responses to chemotherapy. Therefore, determination of GDF11 levels at diagnosis could be a new indicative factor which could help to define groups for alternative therapies (e.g. targeted therapy) instead of serial chemotherapy regimen. In addition, increased GDF11 in patients with favorable prognosis (e.g. CBF, CEBPa, NPM1 mutations) could also be an indication for allogenic BM transplantation. This could reduce toxicity of chemotherapy in patients which do not fit with this therapy and therefore ameliorate patients' survival by reducing undesired toxicity associated with this therapy.

Our studies identify GDF11 as a new soluble factor involved in an autocrine loop controlling chemotherapy resistance in AML. We show that overexpression of GDF11 is correlated with impaired induction chemotherapy responses and in patients with impaired responses to consolidation therapy. Molecular mechanisms involved are still unknown and are currently under investigation in our laboratory. The fact that GDF11 overexpression is not associated with commonly found somatic mutations in NK-AML suggests that GDF11 expression is independent on specific alteration of AML cell biology. Further studies will be necessary to clarify this point but reactive oxygen species (ROS) could be one of the candidates implicated in GDF11 overexpression since we have previously show that ROS induces GDF11 expression and ROS are involved in AML biology^{15,19}. Hence, we propose that GDF11 functions as new regulator of chemotherapy resistance and probably though the modulation of ROS levels which impacts in AML biology. Although our study focused on leukemia, our data imply that the GDF11 overexpression could be involved in chemotherapy resistance of other malignant hematological diseases. We also demonstrated that GDF11 inhibition with blocking antibodies induces apoptosis both in AML cell lines and in primary AML blasts. These preliminary data open major perspectives in the therapy of AML patients.

TABLE 1
Useful nucleotide and amino acid sequences for practicing the invention

| SEQ ID | |
|---|---|
| NO | Nucleotide or amino acid sequence |
| 1 | MVLAAPLLLG FLLLALELRP RGEAAEGPAA AAAAAAAAAA |
| (GDF11 | AGVGGERSSR PAPSVAPEPD GCPVCVWRQH SRELRLESIK |
| AA | SQILSKLRLK EAPNISREVV KQLLPKAPPL QQILDLHDFQ |
| sequence) | GDALQPEDFL EEDEYHATTE TVISMAQETD PAVQTDGSPL |
| | CCHFHFSPKV MFTKVLKAQL WVYLRPVPRP ATVYLQILRL |
| | KPLTGEGTAG GGGGGRRHIR IRSLKIELHS RSGHWQSIDF |
| | KQVLHSWFRQ PQSNWGIEIN AFDPSGTDLA VTSLGPGAEG |
| | LHPFMELRVL ENTKRSRRNL GLDCDEHSSE SRCCRYPLTV |
| | DFEAFGWDWI IAPKRYKANY CSGQCEYMFM QKYPHTHLVQ |
| | QANPRGSAGP CCTPTKMSPI NMLYFNDKQQ IIYGKIPGMV VDRCGCS |
| 2 | tccccgcccc ccagtcctcc ctcccctccc ctccagcatg gtgctcgcgg ccccgctgct gctgggcttc |
| (GDF11 | ctgctcctcg ccctggagct gcggccccgg ggggaggcgg ccgagggccc cgcggcggcg |
| nucleic | gcggcggcgg cggcggcggc ggcagcggcg ggggtcgggg gggagcgctc cagccggcca |
| acid | gccccgtccg tggcgcccga gccggacggc tgccccgtgt gcgtttggcg gcagcacagc |
| sequence) | cgcgagctgc gcctagagag catcaagtcg cagatcttga gcaaactgcg gctcaaggag |
| | gcgcccaaca tcagccgcga ggtggtgaag cagctgctgc ccaaggcgcc gccgctgcag |
| | cagatcctgg acctacacga cttccagggc gacgcgctgc agcccgagga cttcctggag |
| | gaggacgagt accacgccac caccgagacc gtcattagca tggcccagga gacggaccca |
| | gcagtacaga cagatggcag ccctctctgc tgccattttc acttcagccc caaggtgatg ttcacaaagg |
| | tactgaaggc ccagctgtgg gtgtacctac ggcctgtacc ccgcccagcc acagtctacc tgcagatctt |
| | gcgactaaaa cccctaactg gggaagggac cgcaggggga gggggcggag gccggcgtca |
| | catccgtatc cgctcactga agattgagct gcactcacgc tcaggccatt ggcagagcat cgacttcaag |
| | caagtgctac acagctggtt ccgccagcca cagagcaact ggggcatcga gatcaacgcc tttgatccca |
| | gtggcacaga cctggctgtc acctccctgg ggccgggagc cgaggggctg catccattca tggagcttcg |
| | agtcctagag aacacaaaac gttcccggcg gaacctgggt ctggactgcg acgagcactc aagcgagtcc |
| | cgctgctgcc gatatcccct cacagtggac tttgaggctt tcggctggga ctggatcatc gcacctaagc |
| | gctacaaggc caactactgc tccggccagt gcgagtacat gttcatgcaa aaatatccgc atacccattt |
| | ggtgcagcag gccaatccaa gaggctctgc tgggccctgt tgtaccccca ccaagatgtc cccaatcaac |
| | atgctctact tcaatgacaa gcagcagatt atctacggca agatccctgg catggtggtg gatcgctgtg |
| | gctgctctta aggtggggga tagaggatgc ctcccccaca gaccctaccc caagacccct agccctgccc |
| | ccatcccccc aagccctaga gctccctcca ctcttcccgc gaacatcaca ccgttccccg accaagccgt |
| | gtgcaataca acagagggag gcaggtggga attgagggtg aggggtttgg gggaaagggg |
| | aagcaggggc atagtcaggg tggggagtgt ttgaagtttg cagatgagaa ggtttgacaa aaagacagag |
| | agatgtagag acagtgatag agacagagga acaaaaagag cagcagtgag aaggcaaaga |
| | gagaggcaga agagacagac gaggcagaga caaaacactg agaaagagac tgaaatggag |
| | taataaatga aagccccaca ccaagcctcc tttcttccac tggcaaggtg aggggcttgg tatagtttgg |
| | ggagatcccc tgactattca gtaggagaag aaatcaaaaa tccattcttt tctccttctc tccctccaac |
| | agtggccagg ggaaggggaa gtgagggcag gggcaaaaag atttgggaat ttttatttat ttatttattg |
| | tgacttttca tttttttggt atttggcttt actggaatag gagggcccct gcccactgtg ccccgtttat |
| | cccttattcc ccaaaccctg ctctccccaa cacctactca cttaagcact tgtataaagc ctccagggtt |
| | gggaatggga gtaaagggca agagggcgga cacatgaagt ttagtttcta acccatcatc accctaactc |
| | aaccttttct gagccaaatg gcttgaattg aagccagttg tcatggaaat agtaagaggt tagggtttaa |
| | gagctgggga tgcgggggtg ggagagagaa ccctcaacat ccaggatcta tataatgaga gctactttaa |
| | accctcaggt ccaccctcat gatgctgagt tatttagcca gagggtgcag cctgcttatg cccaaattcc |
| | ctcagccaag agagagacca aagagcctct ggaatggccc tgctcccagc ctctatcttc aggtcaatta |
| | gagagagtat agagacccca gagtcccctg ggtctggaaa gcgttaggag aggtcaagaa |
| | aggagcagta aggaggctga aggttacagg gcatttgaat ccaaatcact gctctgggct agggaataga |
| | gccagcagac caaggtggga aggattctgg aagggggaca ttttagtctc ctaaccccaa agctcagggt |
| | ggaagagggg agaacaagga agcagagtgt ataattattt tttcctttta tttttggaat ctaacagtac |
| | ctggcagcag ggaggggaaa gtacagtggg gaaaagcatc tgacaaggcc agttagaaca |
| | gaggatggga aggatggaga ctcccgggct tggaaggcta ggaagcaggc agagactggt |
| | tgccatttca agtcactagc taggcccatt cattcctccc acaaccctga cccattctcc tctggactca |
| | ctgtgcctca gtttcttccc ctcaatggaa tgagaaatga cagcacccgc cacagccaag agatgaattc |
| | tgagcactta ccacgggcac tttatggaca taaaatacct ctcgctgtgg gacagataac cagggcacca |
| | gagtagtggt gaagagatgt gaggcttaag aggagtcaca ggcttcagag tacaagttcc cctctgcctc |
| | ccagctggac agtgcctaga agccaaggag ttgagaatct cctgatccac accctatcct tacttcacca |
| | ccaggcctct tggctccagg caagagctta gaggatgtca ggagaggtgg gggtaagaat cttcagcaaa |
| | actgtcactc taagtagagc cagcagttac gggtctgata aaaacagtac tgaactaaag taaagcccaa |
| | gctggtgagc aaaactggat ggctcattct tcccaagagc atgactctcc cccttggcca gttggtggaa |
| | ggggcaaagg tatgtgacca cccttgagaa ggtgatgttg gtgagcttta acatcttatt cctattctta |
| | tagtgagaaa gtgaaacaag atctttcagt agaggaatgg gcagggctgt taggctcttc agcttgcctt |
| | cacccatata gcagctatgc taaccccaag cctctctggc cctgttcttc atccttcctt ctgccccaat |
| | cctgaaggac aagacacacc cggccatcaa caccactcac atttccttgg tggaaggaaa ggaacagaga |
| | agtgaagaac agatacctcc ctccaaggtc aaatgcctcg tgatcttggc agagtaggga ttgggcaata |
| | agcatcaggt atcttccctctacagattct agagagctgg ggcattaaat atgggggaca cttagaatac |
| | agctccttaa ataccaccaa ataaagacct ttgtgtgtgt gtggtgggtg gggggggggc aggggtcttt |
| | ctcttatgaa cataaatctg tgagctgaag tctcattccc ctgttcctcc ctacccccaa agaggcacag |
| | agtgaaggga cttggggggc acagctcagc aacccagtgg gagttagcac cccctcccac cttatgatgt |
| | gtgtggacct ggccagtgcc cctctgaaca tatcattatt agtgtaatta tcatttattt tgtgtatttg |
| | tcacattgtg tgcatgacag cctttgttaa gggtgtctga ggagtatgga gctgacaggg gcattggaat |
| | gccaggaaag aacttcttcaactgagatca aggcttcctg gagggaacca ctgcaaaaag gccatcaggc |
| | agttttcaag ttatgtgaca gagggcaaag acggccatag ggtgctctga gttttgggat ggtcacatga |
| | cacaatccag cacttgaacc tgaaaaaaaa aataaaagcg gtcaaagagt ttagaattca |
| 3 | MGAAAKLAFAVFLISCSSGAILGRSETQECLFFNANWEKDRTNQTGVE |
| (human | PCYGDKDKRRHCFATWKNISGSIEIVKQGCWLDDINCYDRTDCVEKKD |
| ActRIIA | SPEVYFCCCEGNMCNEKFSYFPEMEVTQPTSNPVTPKPPYYNILLYSLV |
| precursor | PLMLIAGIVICAFWVYRHHKMAYPPVLVPTQDPGPPPPSPLLGLKPLQL |
| polypeptide) | LEVKARGRFGCVWKAQLLNEYVAVKIFPIQDKQSWQNEYEVYSLPGM |
| | KHENILQFIGAEKRGTSVDVDLWLITAFHEKGSLSDFLKANVVSWNEL |
| | CHIAETMARGLAYLHEDIPGLKDGHKPAISHRDIKSKNVLLKNNLTACI |
| | ADFGLALKFEAGKSAGDTHGQVGTRRYMAPEVLEGAINFQRDAFLRID |
| | MYAMGLVLWELASRCTAADGPVDEYMLPFEEEIGQHPSLEDMQEVVV |
| | HKKKRPVLRDYWQKHAGMAMLCETIEECWDHDAEARLSAGCVGERI |
| | TQMQRLTNIITTEDIVTVVTMVTNVDFPPKESSL |
| 4 | MTAPWVALALLWGSLWPGSGRGEAETRECIYYNANWELERTNQSGLE |
| (human | RCEGEQDKRLHCYASWX(AorR)NSSGTIELVKKGCWLDDFNCYDRQEC |
| ActRIIB | VATEENPQVYFCCCEGNFCNERFTHLPEAGGPEVTYEPPPTAPTLLTVL |
| precursor | AYSLLPIGGLSLIVLLAFWMYRHRKPPYGHVDIHEDPGPPPPSPLVGLKP |
| polypeptide) | LQLLEIKARGRFGCVWKAQLMNDFVAVKIFPLQDKQSWQSEREIFSTP |
| | GMKHENLLQFIAAEKRGSNLEVELWLITAFHDKGSLTDYLKGNIITWN |
| | ELCHVAETMSRGLSYLHEDVPWCRGEGHKPSIAHRDFKSKNVLLKSDL |
| | TAVLADFGLAVRFEPGKPPGDTHGQVGTRRYMAPEVLEGAINFQRDAF |
| | LRIDMYAMGLVLWELVSRCKAADGPVDEYMLPFEEEIGQHPSLEELQE |
| | VVVHKKMRPTIKDHWLKHPGLAQLCVTIEECWDHDAEARLSAGCVEE |
| | RVSLIRRSVNGTTSDCLVSL VTSVTNVDLPPKESSI |

### REFERENCES

Throughout this application, various references describe the state of the art to which this invention pertains. The disclosures of these references are hereby incorporated by reference into the present disclosure.
1. Horne, G., Brewin, J. & Chevassut, T. Genetic profiling in acute myeloid leukemia. N Engl J Med 366, 2321; author reply 2321-2322 (2012).
2. Lowenberg, B., Downing, J. R. & Burnett, A. Acute myeloid leukemia. N Engl J Med 341, 1051-1062 (1999).
3. Mardis, E. R., et al. Recurring mutations found by sequencing an acute myeloid leukemia genome. N Engl J Med 361, 1058-1066 (2009).
4. Ferrara, F. & Schiffer, C. A. Acute myeloid leukaemia in adults. Lancet 381, 484-495 (2013).
5. Kantarjian, H., et al. Results of intensive chemotherapy in 998 patients age 65 years or older with acute myeloid leukemia or high-risk myelodysplastic syndrome: predictive prognostic models for outcome. Cancer 106, 1090-1098 (2006).
6. Lowenberg, B., et al. Cytarabine dose for acute myeloid leukemia. N Engl J Med 364, 1027-1036 (2011).
7. Bishop, J. F., et al. A randomized study of high-dose cytarabine in induction in acute myeloid leukemia. Blood 87, 1710-1717 (1996).
8. Hann, I. M., et al. Randomized comparison of DAT versus ADE as induction chemotherapy in children and younger adults with acute myeloid leukemia. Results of the Medical Research Council's 10th AML trial (MRC AML10). Adult and Childhood Leukaemia Working Parties of the Medical Research Council. Blood 89, 2311-2318 (1997).
9. Dohner, H., et al. Diagnosis and management of acute myeloid leukemia in adults: recommendations from an international expert panel, on behalf of the European LeukemiaNet. Blood 115, 453-474 (2010).
10. Ravandi, F., et al. Characteristics and outcome of patients with acute myeloid leukemia refractory to 1 cycle of high-dose cytarabine-based induction chemotherapy. Blood 116, 5818-5823; quiz 6153 (2010).
11. Burnett, A. K., et al. Randomised comparison of addition of autologous bone-marrow transplantation to intensive chemotherapy for acute myeloid leukaemia in first remission: results of MRC AML 10 trial. UK Medical Research Council Adult and Children's Leukaemia Working Parties. Lancet 351, 700-708 (1998).
12. Ferrara, F. Treatment of older patients with acute myeloid leukaemia. Lancet 376, 1967-1968 (2010).
13. Ferrara, F. Conventional chemotherapy or hypomethylating agents for older patients with acute myeloid leukaemia? Hematol Oncol 32, 1-9 (2014).
14. Ferrara, F. Treatment of unfit patients with acute myeloid leukemia: a still open clinical challenge. Clin Lymphoma Myeloma Leuk 11, 10-16 (2011).
15. Dussiot, M., et al. An activin receptor HA ligand trap corrects ineffective erythropoiesis in beta-thalassemia. Nature medicine 20, 398-407 (2014).
16. Suragani, R. N., et al. Modified activin receptor IIB ligand trap mitigates ineffective erythropoiesis and disease complications in murine beta-thalassemia. Blood 123, 3864-3872 (2014).
17. Suragani, R. N., et al. Transforming growth factor-beta superfamily ligand trap ACE-536 corrects anemia by promoting late-stage erythropoiesis. Nature medicine 20, 408-414 (2014).
18. Paulson, R. F. Targeting a new regulator of erythropoiesis to alleviate anemia. Nature medicine 20, 334-335 (2014).
19. Callens, C., Moura, I. C. & Hermine, O. [Targeting oxidative metabolism to treat leukemia?]. Med Sci (Paris) 26, 1033-1035 (2010).

1. A method for predicting the responsiveness of a patient affected with a malignant haematological disease to a chemotherapy treatment, comprising measuring a level of GDF11 in a blood sample from said patient and comparing the level of GDF11 with a control reference value, wherein if the level of GDF11 in the patient is inferior to the control reference value it is concluded that the patient is a responder to the chemotherapy treatment.

2. The method according to claim 1, wherein the malignant haematological disease is acute myeloid leukemia.

3. The method according to claim 1, wherein the chemotherapy treatment is selected from the group consisting of daunorubicin, cytarabine, mitoxantrone, decitabine, and azacitidine.

4. (canceled)

5. A method for monitoring the effectiveness of treatment of a patient affected with a malignant haematological disease to an anti-cancer treatment comprising a step of measuring a level of GDF11 in a blood sample from said patient and comparing the level of GDF11 with a control reference value.

6. The method according to claim 5, wherein the anti-cancer treatment is chemotherapy treatment and/or a GDF11 antagonist.

7-13. (canceled)

14. A pharmaceutical composition comprising a GDF11 antagonist and a pharmaceutically acceptable carrier.

15. The pharmaceutical composition according to claim 14 further comprising an additional therapeutic agent.

16. A method of preventing or treating a malignant haematological disease in a subject comprising administering to the subject a therapeutically effective amount of a GDF11 antagonist.

17. The method of claim 16, wherein the GDF11 antagonist is an inhibitor of the interaction between GDF11 and GDF11 receptor (ActRIIA or ActRIIB).

18. The method according to claim 16, wherein said antagonist is a soluble GDF11 receptor polypeptide.

19. The method according to claim 16, wherein said antagonist is an anti-GDF11 neutralizing antibody or aptamer.

20. The method according to claim 16, wherein said antagonist is an inhibitor of GDF11 gene expression,

21. The method of claim 20, wherein said inhibitor of GDF11 gene expression is a small inhibitory RNA (siRNA), a nuclease, a ribozyme, or an antisense oligonucleotide.

22. The method according to claim 16, wherein the malignant haematological disease is acute myeloid leukemia.

23. The method according to claim 16, wherein the subject suffers from a drug resistant tumor or tumor relapse.

24. A method for treating a patient affected with a malignant haematological disease, comprising
i) identifying the patient as a chemotherapy responder by
measuring a level of GDF11 in a blood sample from said patient,
comparing the level of GDF11 with a control reference value, and
determining that the level of GDF11 is inferior to the control reference value,
and
ii) treating the patient with chemotherapy.


SEQUENCE LISTING (4 sequences)

SEQ ID NO: 1 - protein, 407 aa, Homo sapiens
MVLAAPLLLGFLLLALELRPRGEAAEGPAAAAAAAAAAAAAGVGGERSSRPAPSVAPEPDGCPVCVWRQHSRELRLESIKSQILSKLRLKEAPNISREVVKQLLPKAPPLQQILDLHDFQGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTKRSRRNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 2 - DNA, 4260 bp, Homo sapiens
tccccgccccccagtcctccctcccctcccctccagcatggtgctcgcggccccgctgctgctgggcttcctgctcctcgccctggagctgcggccccggggggaggcggccgagggccccgcggcggcggcggcggcggcggcggcggcggcagcggcgggggtcgggggggagcgctccagccggccagccccgtccgtggcgcccgagccggacggctgccccgtgtgcgtttggcggcagcacagccgcgagctgcgcctagagagcatcaagtcgcagatcttgagcaaactgcggctcaaggaggcgcccaacatcagccgcgaggtggtgaagcagctgctgcccaaggcgccgccgctgcagcagatcctggacctacacgacttccagggcgacgcgctgcagcccgaggacttcctggaggaggacgagtaccacgccaccaccgagaccgtcattagcatggcccaggagacggacccagcagtacagacagatggcagccctctctgctgccattttcacttcagccccaaggtgatgttcacaaaggtactgaaggcccagctgtgggtgtacctacggcctgtaccccgcccagccacagtctacctgcagatcttgcgactaaaacccctaactggggaagggaccgcagggggagggggcggaggccggcgtcacatccgtatccgctcactgaagattgagctgcactcacgctcaggccattggcagagcatcgacttcaagcaagtgctacacagctggttccgccagccacagagcaactggggcatcgagatcaacgcctttgatcccagtggcacagacctggctgtcacctccctggggccgggagccgaggggctgcatccattcatggagcttcgagtcctagagaacacaaaacgttcccggcggaacctgggtctggactgcgacgagcactcaagcgagtcccgctgctgccgatatcccctcacagtggactttgaggctttcggctgggactggatcatcgcacctaagcgctacaaggccaactactgctccggccagtgcgagtacatgttcatgcaaaaatatccgcatacccatttggtgcagcaggccaatccaagaggctctgctgggccctgttgtacccccaccaagatgtccccaatcaacatgctctacttcaatgacaagcagcagattatctacggcaagatccctggcatggtggtggatcgctgtggctgctcttaaggtgggggatagaggatgcctcccccacagaccctaccccaagacccctagccctgcccccatccccccaagccctagagctccctccactcttcccgcgaacatcacaccgttccccgaccaagccgtgtgcaatacaacagagggaggcaggtgggaattgagggtgaggggtttgggggaaaggggaagcaggggcatagtcagggtggggagtgtttgaagtttgcagatgagaaggtttgacaaaaagacagagagatgtagagacagtgatagagacagaggaacaaaaagagcagcagtgagaaggcaaagagagaggcagaagagacagacgaggcagagacaaaacactgagaaagagactgaaatggagtaataaatgaaagccccacaccaagcctcctttcttccactggcaaggtgaggggcttggtatagtttggggagatcccctgactattcagtaggagaagaaatcaaaaatccattcttttctccttctctccctccaacagtggccaggggaaggggaagtgagggcaggggcaaaaagatttgggaatttttatttatttatttattgtgacttttcatttttttggtatttggctttactggaataggagggcccctgcccactgtgccccgtttatcccttattccccaaaccctgctctccccaacacctactcacttaagcacttgtataaagcctccagggttgggaatgggagtaaagggcaagagggcggacacatgaagtttagtttctaacccatcatcaccctaactcaaccttttctgagccaaatggcttgaattgaagccagttgtcatggaaatagtaagaggttagggtttaagagctggggatgcgggggtgggagagagaaccctcaacatccaggatctatataatgagagctactttaaaccctcaggtccaccctcatgatgctgagttatttagccagagggtgcagcctgcttatgcccaaattccctcagccaagagagagaccaaagagcctctggaatggccctgctcccagcctctatcttcaggtcaattagagagagtatagagaccccagagtcccctgggtctggaaagcgttaggagaggtcaagaaaggagcagtaaggaggctgaaggttacagggcatttgaatccaaatcactgctctgggctagggaatagagccagcagaccaaggtgggaaggattctggaagggggacattttagtctcctaaccccaaagctcagggtggaagaggggagaacaaggaagcagagtgtataattattttttccttttatttttggaatctaacagtacctggcagcagggaggggaaagtacagtggggaaaagcatctgacaaggccagttagaacagaggatgggaaggatggagactcccgggcttggaaggctaggaagcaggcagagactggttgccatttcaagtcactagctaggcccattcattcctcccacaaccctgacccattctcctctggactcactgtgcctcagtttcttcccctcaatggaatgagaaatgacagcacccgccacagccaagagatgaattctgagcacttaccacgggcactttatggacataaaatacctctcgctgtgggacagataaccagggcaccagagtagtggtgaagagatgtgaggcttaagaggagtcacaggcttcagagtacaagttcccctctgcctcccagctggacagtgcctagaagccaaggagttgagaatctcctgatccacaccctatccttacttcaccaccaggcctcttggctccaggcaagagcttagaggatgtcaggagaggtgggggtaagaatcttcagcaaaactgtcactctaagtagagccagcagttacgggtctgataaaaacagtactgaactaaagtaaagcccaagctggtgagcaaaactggatggctcattcttcccaagagcatgactctcccccttggccagttggtggaaggggcaaaggtatgtgaccacccttgagaaggtgatgttggtgagctttaacatcttattcctattcttatagtgagaaagtgaaacaagatctttcagtagaggaatgggcagggctgttaggctcttcagcttgccttcacccatatagcagctatgctaaccccaagcctctctggccctgttcttcatccttccttctgccccaatcctgaaggacaagacacacccggccatcaacaccactcacatttccttggtggaaggaaaggaacagagaagtgaagaacagatacctccctccaaggtcaaatgcctcgtgatcttggcagagtagggattgggcaataagcatcaggtatcttccctctacagattctagagagctggggcattaaatatgggggacacttagaatacagctccttaaataccaccaaataaagacctttgtgtgtgtgtggtgggtggggggggggcaggggtctttctcttatgaacataaatctgtgagctgaagtctcattcccctgttcctccctacccccaaagaggcacagagtgaagggacttggggggcacagctcagcaacccagtgggagttagcaccccctcccaccttatgatgtgtgtggacctggccagtgcccctctgaacatatcattattagtgtaattatcatttattttgtgtatttgtcacattgtgtgcatgacagcctttgttaagggtgtctgaggagtatggagctgacaggggcattggaatgccaggaaagaacttcttcaactgagatcaaggcttcctggagggaaccactgcaaaaaggccatcaggcagttttcaagttatgtgacagagggcaaagacggccatagggtgctctgagttttgggatggtcacatgacacaatccagcacttgaacctgaaaaaaaaaataaaagcggtcaaagagtttagaattca

SEQ ID NO: 3 - protein, 513 aa, Homo sapiens
MGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPYYNILLYSLVPLMLIAGIVICAFWVYRHHKMAYPPVLVPTQDPGPPPPSPLLGLKPLQLLEVKARGRFGCVWKAQLLNEYVAVKIFPIQDKQSWQNEYEVYSLPGMKHENILQFIGAEKRGTSVDVDLWLITAFHEKGSLSDFLKANVVSWNELCHIAETMARGLAYLHEDIPGLKDGHKPAISHRDIKSKNVLLKNNLTACIADFGLALKFEAGKSAGDTHGQVGTRRYMAPEVLEGAINFQRDAFLRIDMYAMGLVLWELASRCTAADGPVDEYMLPFEEEIGQHPSLEDMQEVVVHKKKRPVLRDYWQKHAGMAMLCETIEECWDHDAEARLSAGCVGERITQMQRLTNIITTEDIVTVVTMVTNVDFPPKESSL

SEQ ID NO: 4 - protein, 512 aa, Homo sapiens
  VARIANT (64)..(64): X is A or R
MTAPWVALALLWGSLWPGSGRGEAETRECIYYNANWELERTNQSGLERCEGEQDKRLHCYASWXNSSGTIELVKKGCWLDDFNCYDRQECVATEENPQVYFCCCEGNFCNERFTHLPEAGGPEVTYEPPPTAPTLLTVLAYSLLPIGGLSLIVLLAFWMYRHRKPPYGHVDIHEDPGPPPPSPLVGLKPLQLLEIKARGRFGCVWKAQLMNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLPPKESSI